(19) United States
(12) Patent Application Publication
(10) Pub. No.: US 2008/0200527 A1
(43) Pub. Date: Aug. 21, 2008

(54) Cannabinoid Receptor Modulators

(75) Inventors: Remi Alain Amengual, Le Raincy (FR); Claire Nadelene Marsol, Avolsheim (FR); Eric Liu Mayeux, Eschau (FR); Michael laurence Sierra, Soeborg (DK); Patrick Herve Wagner, Brumath (FR)

Correspondence Address: BANNER & WITCOFF, LTD., 1100 13th STREET, N.W., SUITE 1200, WASHINGTON, DC, 20005-4051, US

(73) Assignee: Carex SA, F-111Kirch, Cedex (FR)

(21) Appl. No.: 11/917,782

(22) PCT Filed: Jun. 14, 2006
(86) PCT No.: PCT/EP06/05726

§ 371 Date: Dec. 17, 2007

(30) Foreign Application Priority Data
Jun. 17, 2005 (EP) 05360022.7
Sep. 26, 2005 (EP) 05360032.6
Nov. 24, 2005 (EP) 05360047.4

(51) Int. Cl. A61K 31/41; C07D 403/12; C07D 231/14; A61K 31/415; A61P 3/00; A61P 3/04

(52) U.S. Cl. 514/381; 548/253; 548/374.1; 514/406

(57) ABSTRACT

Compounds of formula (I), are cannabinoid CB1 receptors, useful, inter alia in the treatment of obesity:

wherein A₁ is hydrogen, —COOH, or tetrazolyl, and A₂ is hydrogen, —COOH, tetrazolyl, —CN, —CF₃, —COR₆, —SO₂R₆, —OR₇, —NR₇R₈, —NHCOR₆, and —NR₇SO₂R₈ provided that one of A₁ and A₂ is either —COOH or tetrazolyl; p is 0 or 1 and A₃ is phenyl or cycloalkyl, either of which is optionally substituted with R₄ and/or R₅; q is 0 or 1; R₁ is a bond, or —(CH₂)ₐB₁(CH₂)_{b}— wherein a and b are independently 0, 1, 2 or 3 provided that a+b is not greater than 4, and B₁ is —CO—, —O—, —S—, —SO—, —SO₂—, —CH₂—, —CHOH— or —NR₇—; R₂ is a bond, —CH₂)ₐB₁(CH₂)_{b}— or —[(CH₂)ₐB₁(CH₂)_{b}]ₙ-A₄-[(CH₂)_{c}B₂(CH₂)_{d}]ₘ— wherein a, b, and B₁ are as defined for R₁; B₂ is as defined for B₁, c and d are independently 0, 1, 2 or 3; with the proviso that a+b+c+d is not greater than 6, n and m are independently 0 or 1 and A₄ is a monocarbocyclic or monoheterocyclic ring, having 3 to 8 ring atoms, optionally substituted with one or more of —F, —Cl, —Br, —CN, —CF₃, C₁-C₄ alkyl, cycloalkyl, —OR₉, oxo or —NR₇R₈; R₃ is hydrogen, C₁-C₄ alkyl, cycloalkyl, —CF₃, —OR₉, —NR₇R₈, —(CH₂)ₛCOR₆, —(CH₂)ₛSO₂R₆, —(CH₂)ₛNR₇COR₆, —(CH₂)ₛNR₇COOR₈, —(CH₂)ₛNR₇SO₂R₆, wherein s is 1, 2, 3 or 4; R₄ and R₅ independently —R₉, —CN, —F, —Cl, —Br, —OR₉, —NR₇R₈, —NR₇COR₆, —NR₇SO₂R₆, —COR₆, —SR₉, —SOR₉, —SO₂R₆, (C₁-C₄ alkyl)OR₉, —(C₁-C₄ alkyl)NR₇R₈, —(C₁-C₄ alkyl)NR₇COR₆, C₁-C₄ alkyl)NR₇COOR₈, —(C₁-C₄ alkyl)NR₇SO₂R₆, —(C₁-C₄ alkyl)COR₆, —(C₁-C₄ alkyl)SO₂R₆, —NR₇COOR₈, or [N—(C₁-C₄ alkyl)]-tetrazolyl; R₆ is C₁-C₄ alkyl, cycloalkyl, —CF₃ or —NR₇R₈; R₇ and R₈ are independently hydrogen, C₁-C₄ alkyl or cycloalkyl; and R₉ is hydrogen, C₁-C₄ alkyl, cycloalkyl, fully or partially fluorinated C₁-C₄ alkyl.

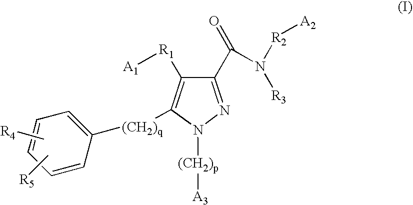

The present invention relates to compounds which are ligands of cannabinoid receptor CB1 and which suppress the normal signalling activity of such receptors. The invention further relates to compositions and methods using said compounds for the treatment of obesity and overweight, and diseases for which obesity is a risk factor, such as metabolic syndrome, Type II diabetes, cardiovascular disease, osteoarthritis, and some cancers; mental disorders; sexual dysfunctions; reproductive dysfunctions; and epilepsy. The invention also relates to pharmaceutical compositions containing the compounds of the invention, and to the use of the compounds in combination with other treatments for overweight- and obesity-dependent diseases.

### BACKGROUND TO THE INVENTION

The prevalence of obesity has risen significantly in the past decade in the United States and many other developed countries. Obesity is now recognized as a chronic disease (Fiegal et al, 1998, Int. J. Obesity 22:39-47, Mokdad et al, 1999, JAMA 282:1519-1522). The “identifiable signs and symptoms” of obesity include an excess accumulation of fat or adipose tissue, an increase in the size or number of fat cells (adipocyte differentiation), insulin resistance, increased glucose levels (hyperglycemia), increased blood pressure, elevated cholesterol and triglyceride levels, decreased levels of high-density lipoprotein and norepinephrine. Because obesity is associated with a significantly elevated risk for type 2 diabetes, coronary heart disease, strokes, hypertension, and numerous other major illnesses, and overall mortality from all causes (Must et al, 1999, JAMA 282:1523-1529, Calle et al, 1999, N. Engl. J. Med. 341:1097-1105), weight reduction is critical for the obese patient (Blackburn, 1999, Am. J. Clin. Nujtr. 69:347-349, Galuska et al, 1999, JAMA 282:1576) as it can improve cardiovascular and metabolic values to reduce obesity-related morbidity and mortality. It has been shown that 5-10% loss of body weight can substantially improve metabolic parameters, such as blood glucose, blood pressure, and lipid concentrations.

Thus, the primary aim of treatment for obesity is weight loss. Initially, treatments have been proposed which were based on diet and lifestyle changes augmented by therapy with oral pharmacological therapies. However, while physical exercise and reductions in dietary intake of calories can improve the obese condition, compliance with this treatment is very poor because of sedentary lifestyles and excess food consumption, especially high fat containing food. Additionally, treatment with the available pharmacological therapies to facilitate weight loss fail to provide adequate benefit to many obese patients because of side effects, contraindications or lack of positive response. Hence, there is impetus for developing new and alternative treatments for management of obesity.

Currently developed drugs effective in obesity treatment act by different mechanisms such as reduction in food intake (e.g. by inducing satiety), drugs altering metabolism (such as agents modifying the absorption of nutrients e.g. inhibition of fat absorption), drugs that increase energy expenditure (e.g. increase of thermogenesis) or that stimulate adipocyte apoptosis.

At present, only two drugs are marketed for obesity treatment (for a review, see Weigle, 2003, J Clin Endocrinol Metab., 88, 2462-2469). Sibutramine is drug acting on the central nervous system by inhibiting serotonin and norepinephrine presynaptic re-uptake. Its primary mechanism of action is increased satiety, although some evidence also suggests increased energy expenditure could play a role in sibutramine-induced weight loss (Poston and Foreyt, 2004, Expert. Opin. Pharmacother., 5, 633-642). Its main side effects are hypertension, tachycardia, headache, insomnia, dry mouth and constipation. Orlistat is an inhibitor of gastrointestinal lipases, predominantly pancreatic lipase, required for the hydrolysis of triglyceride to free fatty acids in the lumen of the gut. As orlistat is able to block the absorption of dietary fat by up to 30%, it produces weight loss comparable to or greater than that obtained by placing an individual on a fat-restricted diet. Interference with vitamin absorption, loose stools, increased defecation, faecal urgency, oily discharge are its main side effects, and may lead to discontinuation of the drug.

Besides, several anti-obesity agents are currently investigated (for a review, see Bays, 2004, Obesity Research, 12, 1197-1211) such as i) central nervous system agents that affect neurotransmitters or neural ion channels (e.g. bupropion, GW320659, topiramate, zonisamide); ii) leptin/insulin/central nervous system pathway agents (e.g. leptin analogues, leptin transport and/or leptin receptor promoters, Axokine, neuropeptide Y antagonists); iii) gastrointestinal-neural pathway agents (e.g. extendin 4, liraglutide, dipeptidyl peptidase IV inhibitors, pramlintide); iv) agents that may increase resting metabolic rate; and v) other more diverse agents, such as for example including melanin concentrating hormone antagonists, amylase inhibitors, antagonists of adipocyte 11B-hydroxysteroid dehydrogenase type 1 activity (for a review, see Bays, 2004, Obesity Research, 12, 1197-1211).

Presently, two cannabinoid receptors have been characterized: CB1, a receptor found in the mammalian brain and in a number of other sites in peripheral tissues; and CB2, a peripheral receptor found principally in cells related to the immune system. For a review on cannabinoid CB1 and CB2 receptor modulators, see Pertwee, 2000, Exp. Opin. Invest. Drugs, 9, 1553-1571. A substantial body of evidence indicates that CB1 antagonists (e.g. SR141716 (Rimonabant)) are able to modulates energy homeostasis and that CB1 antagonists are able to modulate food intake as well as peripherally block lipogenic processes (Cota et al. et al., 2003, J. Clinical Investigation, 112, 423-431). Thus, the cannabinoid system has been shown to be an essential endogenous regulator of energy homeostasis, and represents a target for the development of therapeutic compounds likely to have utility in the treatment and/or prevention of diseases that involve the CB1 receptor, e.g. diseases characterized by impaired energy balance. Literature (for a review, see Reggio, 2003, Current Pharmaceutical Design, 9, 1607-1633 or Lange and Kruse, 2004, Current Opinion in Drug Discovery & Dev., 7, 498-506) refers to several CB1 antagonists, but while the responses observed are encouraging, they are not fully satisfactory because of the occurrence of undesirable side effects, possibly associated with their central effects, also called psychoactive effects, such as for example anxiety, depression, nausea, headaches and vomiting. Accordingly, there still exists a need for alternative CB1 antagonists.

### BRIEF DESCRIPTION OF THE INVENTION

The present invention makes available a class of compounds which act on the cannabinoid receptor CB1. The compounds of the invention are therefore capable of modulating body weight and energy consumption to maintain metabolism and body composition in mammals. The extent to which the compounds modulate the peripheral CB1 receptors, as opposed to the CB1 receptors present in the brain varies from compound to compound. Those compounds of the invention which primarily act on the peripheral CB1 receptors have a reduced propensity to induce psychoactive effects, such as for example anxiety, depression, nausea, headaches and vomiting.

### DETAILED DESCRIPTION OF THE INVENTION

According to the invention, there is provided a compound of formula (I), or a salt, hydrate, solvate or N-oxide thereof:

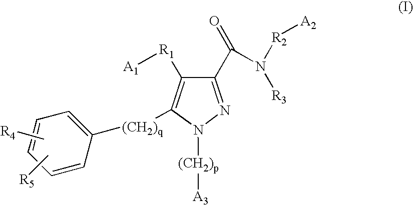

wherein

A₁ is hydrogen, —COOH, or tetrazolyl, and A₂ is hydrogen, —COOH, tetrazolyl, —CN, —CF₃, —COR₆, —SO₂R₆, —OR₇, —NR₇R₈, —NHCOR₆, or —NR₇SO₂R₈ provided that one of A₁ and A₂ is either —COOH or tetrazolyl;

p is 0 or 1 and A₃ is phenyl or cycloalkyl, either of which is optionally substituted with R₄ and/or R₅;
q is 0 or 1;
R₁ is a bond, or —(CH₂)ₐB₁(CH₂)_{b}— wherein a and b are independently 0, 1, 2 or 3 provided that a+b is not greater than 4, and B₁ is —CO—, —O—, —S—, —SO—, —SO₂—, —CH₂—, —CHOH— or —NR₇—;
R₂ is a bond, —(CH₂)ₐB₁(CH₂)_{b}— or —[(CH₂)ₐB₁(CH₂)_{b}]ₙ-A₄-[(CH₂)_{c}B₂(CH₂)_{d}]ₘ— wherein a, b, and B₁ are as defined for R₁; B₂ is as defined for B₁, c and d are independently 0, 1, 2 or 3; with the proviso that a+b+c+d is not greater than 6, n and m are independently 0 or 1 and A₄ is a monocarbocyclic or monoheterocyclic ring, having 3 to 8 ring atoms, optionally substituted with one or more of —F, —Cl, —Br, —CN, —CF₃, C₁-C₄ alkyl, cycloalkyl, —OR₉, oxo or —NR₇R₈;
R₃ is hydrogen, C₁-C₄ alkyl, cycloalkyl, —CF₃, —OR₉, —NR₇R₈, —(CH₂)ₛCOR₆, —(CH₂)ₛSO₂R₆, —(CH₂)ₛNR₇COR₆, —(CH₂)ₛNR₇COOR₈, —(CH₂)ₛNR₇SO₂R₆, wherein s is 1, 2, 3 or 4;
R₄ and R₅ independently —R₉, —CN, —F, —Cl, —Br, —OR₉, —NR₇R₈, —NR₇COR₆, —NR₇SO₂R₆, —COR₆, —SR₉, —SOR₉, —SO₂R₆, (C₁-C₄ alkyl)OR₉, —(C₁-C₄ alkyl)NR₇R₈, —(C₁-C₄ alkyl)NR₇COR₆, C₁-C₄ alkyl)NR₇COOR₈, —(C₁-C₄ alkyl)NR₇SO₂R₆, —(C₁-C₄ alkyl)COR₆, —(C₁-C₄ alkyl)SO₂R₆, —NR₇COOR₈, or [N—(C₁-C₄ alkyl)]-tetrazolyl;
R₆ is C₁-C₄ alkyl, cycloalkyl, —CF₃ or —NR₇R₈;
R₇ and R₈ are independently hydrogen, C₁-C₄ alkyl or cycloalkyl and
R₉ is hydrogen, C₁-C₄ alkyl, cycloalkyl, fully or partially fluorinated C₁-C₄ alkyl.

As used herein the term “salt” includes base addition, acid addition and quaternary salts. Compounds of the invention which are acidic can form salts, including pharmaceutically or veterinarily acceptable salts, with bases such as alkali metal hydroxides, e.g. sodium and potassium hydroxides; alkaline earth metal hydroxides e.g. calcium, barium and magnesium hydroxides; with organic bases e.g. N-ethyl piperidine, dibenzylamine and the like. Those compounds (I) which are basic can form salts, including pharmaceutically or veterinarily acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic and p-toluene sulphonic acids and the like.

The term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl. It is also intended that the term shall include straight or branched chain alkyl radicals having from a to b carbon atoms and which contain double or triple bond such as, example, vinyl, allyl, 1- and 2-butenyl and 2-methyl-2-propenyl, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl.

The term “fully or partially fluorinated Cₐ-C_{b} alkyl wherein a and b are integers refers to a Cₐ-C_{b} alkyl radical in which at least one hydrogen is replaced by fluoro. Such radicals include, for example, —CF₃, —CH₂F, —CF₂H and —CH₂CF₃.

As used herein the unqualified term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

The compounds of the invention may contain one or more chiral centres, because of the presence of asymmetric carbon atoms, and they may therefore exist as a number of diastereoisomers with R or S stereochemistry at each chiral centre. The invention includes all such diastereoisomers and mixtures thereof.

For use in accordance with the invention, the following structural characteristics are currently preferred, in any compatible combination, in the compounds (I):

### The Radical A₃(CH₂)ₚ

p may be 1 or 0, but currently it is preferred that p be 0.
Any optional R₄ and/or R₅ substituents in A₃ are preferably selected from —Cl, —F, —Br, —CN, —CF₃, C₁-C₄ alkyl such as methyl, ethyl or iso-propyl, cycloalkyl such as cyclopropyl, cyclopentyl or cyclohexyl, —O(C₁-C₄ alkyl) such as methoxy or ethoxy, —O(cycloalkyl) such as cyclopropyloxy, —SO₂(C₁-C₄ alkyl), such as methylsulfonyl, ethylsulfonyl or iso-prpylsulfonyl, —SO₂NH(cycloalkyl) such as cyclopropylaminosulfonyl, —SO₂NH(C₁-C₄ alkyl) such as methylaminosulfonyl, —SCF₃ and —OCF₃.
A₃ is preferably a phenyl ring, which may be substituted in the ortho position and/or the para position by —Cl, —CF₃, —OCF₃, —SCF₃, —F, —Br, or —CN.

### The Radical R₄R₅Ph(CH₂)_{q}

q may be 1 or 0, but currently it is preferred that q be 0.
Any optional R₄ and/or R₅ substituents in the phenyl ring are preferably selected from —Cl, —F, —Br, —CN, —CF₃, C₁-C₄ alkyl such as methyl, ethyl or iso-propyl, cycloalkyl such as cyclopropyl, cyclopentyl or cyclohexyl, —O(C₁-C₄ alkyl) such as methoxy or ethoxy, —O(cycloalkyl) such as cyclopropyloxy, —OCF₃, and —SO₂R₆ and —OR₉ or —SR₉ wherein R₆ is C₁-C₄ alkyl such as methyl, ethyl or iso-propyl or cycloalkyl such as cyclopropyl, cyclopentyl or cyclohexyl. In one subset of the compounds of the invention R₄ is hydrogen and R₅ is a substituent in the para position selected from the foregoing, particularly —Cl, —F, —Br, —CN, —CF₃, C₁-C₄ alkyl, cycloalkyl, —O(C₁-C₄ alkyl), —SO₂(C₁-C₄ alkyl), —O(cycloalkyl), —SO₂NH₂, —SO₂NHCH₃, —SO₂N(CH₃)₂, and —OCF₃.

### The Radicals A₂R₂N(R₃)CO— and A₁R₁—

R₃ may be, for example, hydrogen, methyl or methoxy, but hydrogen is presently preferred.
In a first subset of compounds of the invention A₂ is tetrazolyl or, preferably, —COOH, and A₁ is hydrogen. In this subset R₂ is preferably bond, or —(CH₂)₁₋₅—, or the radical —R₂A₂ is

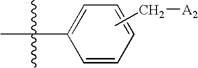

preferably with the A₂CH₂— radical in the 4-position of the phenyl ring.
Also in this first subset, it is currently preferred that R₁ is —CH₂—. It is also currently preferred that A₁ is hydrogen.
In a second subset of compounds of the invention, A₁ is tetrazolyl or, preferably, —COOH, and A₂ is hydrogen, —COR₆, —OR₇, —NR₇R₈, —NHCOR₆, —CN, —CF₃, —SO₂N(R₇)(R₈), —NR₇SO₂CF₃, or —NR₇SO₂R₇ wherein R₆, R₇, and R₈ are as defined with reference to formula (I) above. For example A₂ may be hydrogen, —COCH₃, —CONH₂, —OCH₃ or —NHSO₂butyl. In this second subset, R₁ may be —(CH₂)ₐB₁(CH₂)_{b}— wherein a and b are each 1 and B₁ is as defined with reference to formula (I), but currently it is preferred that R₁ be —CH₂— or —OCH₂—.
This second subset of compounds of the invention includes those wherein —R₂A₂ is —O(CH₂)CO₂H, and also those of formula (IA):

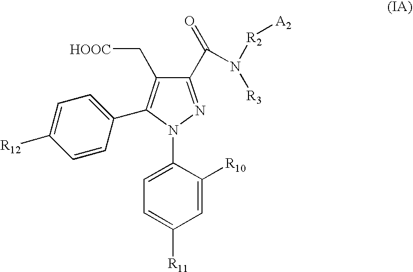

wherein R₁₀ is —Cl, —CF₃, —OCF₃, —F, —Br, or —CN; R₁₁ is hydrogen, —Cl, —CF₃, —OCF₃, —F, —Br, or —CN; R₁₂ is —Cl, —CF₃, —OCF₃, —F, —Br, —OCH₃, —O(cyclopropyl), —SO₂NH₂, or —CN; and A₂, R₂ and R₃ are as defined and discussed above.
In compounds of the second subset, including those of formula (IA), when A₂ is not hydrogen, any R₆, R₇, and R₈ present in A₂ are preferably independently selected hydrogen, methyl, —SO₂NH₂, —SO₂NHCH₃, —CONHCH₃, 2-oxo-pyrrolidin-1-yl, or 2-oxo-piperidin-1-yl.
In specific cases of this second subset A₂, may be, for example —CONH₂, —OCH₃ or —NHSO₂CH₃.
Also in compounds of the second subset, including those of formula (IA), R₂ may be, for example, a bond or —(CH₂)₁₋₅— or the radical —R₂A₂ may be

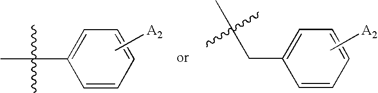

preferably with the A₂- radical in the 4-position of the phenyl ring.
Also in compounds of the second subset R₂ may be

[(CH₂)ₐB₁(CH₂)_{b}]ₙ-A₄-[(CH₂)_{c}B₂(CH₂)_{d}]ₘ—
wherein a, b, c, d, n, m, B₁ and B₂ are as defined and discussed above and A₄ is (i) a divalent piperidine, piperazine, piperazine optionally substituted by methyl on one of the ring nitrogens, morpholine, cyclopropane, cyclobutane, cyclopentane, cyclohexane, cycloheptane, or 2-oxo-pyrrolidin-2-yl radical, or (ii) a divalent phenylene or monocyclic heteroarylene radical having from 5 or 6 ring atoms (such as a divalent pyridine, pyrimidine, thiophene or furan radical), optionally substituted with R₄ and/or R₅ as defined and discussed above. In either case (i) or (ii), A₂ may be, for example, hydrogen. In either case (i) or (ii), both m and n may be 0 or 1, or one of m and n may be 1 and the other 0. B₁ and/or B₂ when present may be —CH₂—, —O—, —S— or —NH—, while a and b, and/or c and d, are each 0 or while a is 1 or 2 and b is 1 or 2, and/or c is 1 or 2 and d is 1 or 2 In a specific case, n may be 1 while m is 0, B₁ may be a bond, a+b may be 0, 1, 2 or 3, and m may be 0 or 1. In another specific case, n and m may both be 1, B₁ and B₂ may each be a bond, a+b may be 0, 1, 2 or 3, and c+d may be 0, 1, 2 or 3.
Specific examples of the radicals A₂R₂N(R₃)CO— and A₁R₁— which may be present in compounds of the invention include those present in the compounds of the Examples herein.

Compounds of the invention include those of the Examples herein, in particular the following, and their salts hydrates solvates, and where feasible their N-oxides:
[5-(4-Chloro-phenyl)-1-(2-chloro-phenyl)-3-(4-cyano-benzylcarbamoyl)-1H-pyrazol-4-yl]-acetic acid,
[5-(4-Chloro-phenyl)-1-(2,4-dichloro-phenyl)-3-(4-cyano-benzylcarbamoyl)-1H-pyrazol-4-yl]-acetic acid,
[5-(4-Chloro-phenyl)-1-(2-chloro-phenyl)-3-(4-cyano-2-fluoro-benzylcarbamoyl)-1H-pyrazol-4-yl]-acetic acid,
(1-(2-Chloro-phenyl)-5-(4-chloro-phenyl)-3-[(5-cyano-thiophen-2-ylmethyl)-carbamoyl]-H-pyrazol-4-yl)-acetic acid.
[5-(4-Chloro-phenyl)-3-(4-cyano-benzylcarbamoyl)-1-(2-fluoro-phenyl)-1H-pyrazol-4-yl]-acetic acid,
[5-(4-Chloro-phenyl)-1-(2-chloro-phenyl)-3-(4-cyano-phenylcarbamoyl)-1H-pyrazol-4-yl]-acetic acid,
[5-(4-Chloro-phenyl)-1-(2-chloro-phenyl)-3-(3-cyano-phenylcarbamoyl)-1H-pyrazol-4-yl]-acetic acid,
[5-(4-Chloro-phenyl)-1-(2-chloro-phenyl)-3-(4-fluoro-phenylcarbamoyl)-1H-pyrazol-4-yl]-acetic acid and
5-(4-Chloro-phenyl)-1-(2,4-dichloro-phenyl)-4-(2H-tetrazol-5-ylmethyl)-1H-pyrazole-3-carboxylic acid 4-cyano-benzylamide.

Preferred compounds of the present invention act primarily on peripheral cannabinoid receptor CB1. Such compounds concentrate primarily in physiological systems and components external to the nervous system, and more particularly external to the central nervous system, i.e. the compound does not readily cross the blood-brain barrier.

The compounds of the invention modulate the cannabinoid receptor CB1 by suppressing its natural signalling function. The compounds are therefore CB1 receptor antagonists, inverse agonists, or partial agonists.

The term “CB1 antagonist” or “cannabinoid receptor CB1 antagonist” refers to a compound which binds to the receptor, or in its vicinity, and lacks any substantial ability to activate the receptor itself. A CB1 antagonist can thereby prevent or reduce the functional activation or occupation of the receptor by a CB1 agonist such as for example the endogenous agonist N-Arachidonylethanolamine (anandamide). This term is well known in the art.

The term “CB1 inverse agonist” or “cannabinoid receptor CB1 inverse agonist” refers to a compound which binds to the receptor and exerts the opposite pharmacological effect as a CB1 receptor agonist does. Inverse agonists are effective against certain types of receptors which have intrinsic activity without the acting of a ligand upon them (also referred to as ‘constitutive activity’). This term is well known in the art. It is also well known in the art that such a CB1 inverse agonist can also be named a CB1 antagonist as the general properties of both types are equivalent. Accordingly, in the context of the present invention the term “CB1 antagonist” in general is understood as including both the “CB1 antagonist” as defined above and the “CB1 inverse agonist”.

The term “CB1 partial agonist” or “cannabinoid receptor CB1 partial agonist” refers to a compound which acts upon the same receptor as the full agonist but that produces a weak maximum pharmacological response and has a low level of intrinsic activity. This term is well known in the art.

According to a preferred embodiment of the present invention, the “CB1 modulator” or “cannabinoid receptor CB1 modulator” is a CB1 antagonist or inverse agonist compound.

In many cases, e.g in the case where A₁ or A₂ is —COOH, the compounds may be administered in a prodrug form. A prodrug is in most cases a pharmacologically inactive derivative of a parent drug molecule that requires spontaneous or enzymatic transformation within the body in order to release the active drug, and that has improved delivery properties over the parent drug molecule. Prodrugs include acid derivatives well known to practitioners of the art, such as, for example, alkyl esters prepared by reaction of the parent acid compound with a suitable alcohol, or amides prepared by reaction of the parent acid compound with a suitable amine. For example, ester prodrugs include methyl, ethyl, propyl, isopropyl, n-butyl, isobutyl, tert-butyl, morpholinoethyl, and N,N-diethylglycolamido esters

The present invention further concerns pharmaceutical compositions comprising at least one of the compound of the Invention associated with a pharmaceutically acceptable excipient which may be a carrier or a diluent or be diluted by a carrier, or enclosed within a carrier which can be in the form of a capsule, sachet, paper, tablets, aerosols, solutions, suspensions or other container. In making the combination products, conventional techniques for the preparation of pharmaceutical compositions may be used. For example, the active compounds will usually be mixed with a carrier or a diluent, or diluted by a carrier or a diluent, or enclosed within a carrier or a diluent which may be in the form of a ampoule, capsule, sachet, paper, tablets, aerosols, solutions, suspensions or other container. When the carrier serves as a diluent, it may be solid, semi-solid, or liquid material which acts as a vehicle, excipient, or medium for the active compound. The active compounds can be adsorbed on a granular solid container for example in a sachet. Typically, liquid oral pharmaceutical compositions are in the form of, for example, suspensions, elixirs and solutions; solid oral pharmaceutical compositions are in the form of, for example, powders, capsules, caplets, gelcaps and tablets. Because of their ease in administration, tablets and capsules represent the most advantageous oral dosage unit form, in which case solid pharmaceutical carriers are obviously employed.

Some examples of suitable carriers or diluents are, without being limited, water, salt solutions, alcohols, polyethylene glycols, polyhydroxyethoxylated castor oil, peanut oil, olive oil, gelatine, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone.

Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavouring agents. The formulations of the invention may be formulated so as to provide quick, sustained, or delayed release of the active ingredient after administration to the patient by employing procedures well known in the art.

In one embodiment, the active compound is prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. The compound of the present invention can also be administered in the form of liposome delivery systems, such as small unilamellar vesicles, large unilamellar vesicles, and multilamellar vesicles. Liposomes can be formed from a variety of lipids, including but not limited to amphipathic lipids such as phosphatidylcholines, sphingomyelins, phosphatidylethanolamines, phophatidylcholines, cardiolipins, phosphatidylserines, phosphatidylglycerols, phosphatidic acids, phosphatidylinositols, diacyl trimethylammonium propanes, diacyl dimethylammonium propanes, and stearylamine, neutral lipids such as triglycerides, and combinations thereof. They may either contain cholesterol or may be cholesterol-free. These can be prepared according to methods known to those skilled in the art, for example, as described in US Pat. N° 4,522,811. The pharmaceutical compositions of the invention can be sterilized and mixed, if desired, with auxiliary agents, emulsifiers, salt for influencing osmotic pressure, buffers and/or colouring substances and the like, which do not deleteriously react with the active compounds.

Hence, additional aspects of the invention include

(i) use of a compound of the invention in the preparation of a composition for treatment of diseases or pathologic conditions associated with metabolic disorders mediated by or related to CB1 mediated mechanisms; and
(ii) a method for the treatment of treatment of diseases or pathologic conditions associated with metabolic disorders mediated by or related to CB1 mediated mechanisms, which method comprises administering to a subject suffering such disease or condition an effective amount of a compound of formula (I) as discussed and defined herein.

The disease or condition treated may be obesity or overweigh, or a diseases or condition related to obesity or overweight. Diseases or condition related to obesity or overweight include those associated with impaired metabolism of glucose, cholesterol and/or triglycerides, such as insulin resistance, Type II diabetes, Type I diabetes, impaired glucose tolerance, dyslipidemia, hyperlipidemia, hypercholesterolemia, hypertriglycidemia, Syndrome X including hypertension, obesity, hyperglycaemia, atherosclerosis, coronary artery disease, heart failure and other cardiovascular disorders, stroke, gout, osteoarthritis, reduced fertility and many other psychological and social problems.

Insulin resistance is manifested by the diminished ability of insulin to exert its biological action across a broad range of concentrations. During early stages of insulin resistance, the body secretes abnormally high amounts of insulin to compensate for this defect. Within developed countries, diabetes mellitus has become a common problem and is associated with a variety of abnormalities including, but not limited to, obesity, hypertension, hyperlipidemia and renal complications. It is now increasingly being recognized that insulin resistance and hyperinsulinemia contribute significantly to obesity, hypertension, atherosclerosis and Type II diabetes mellitus. The association of insulin resistance with obesity, hypertension and angina pectoris has been described as a syndrome (Syndrome-X) in which insulin resistance plays the central role.

Hyperlipidemia is considered the primary cause of cardiovascular and other peripheral vascular diseases. An increased risk of cardiovascular disease is correlated with elevated plasma levels of LDL (Low Density Lipoprotein) and VLDL (Very Low Density Lipoprotein) as seen in hyperlipidemia. Numerous studies have shown that lowering of plasma triglycerides and total cholesterol, in particular LDL and VLDL and increasing HDL cholesterol leads to a significant reduction of cardiac events.

Osteoporosis, is another disease associated with obesity and overweight and the present invention further concerns a method for treating osteoporosis in a patient in need thereof wherein said method comprises the administration to said patient of at least one CB1 modulator compound of the Invention, and more particularly acting primarily on peripheral cannabinoid receptor CB1 (see Idris et al., 2005, Nature Medicine, May 22, doi:10.1038/nm1255).

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial, as is required in the pharmaceutical art. However, for administration to human patients, the total daily dose of the compounds of the invention may typically be in the range 1 mg to 1000 mg depending, of course, on the mode of administration. For example, oral administration may require a total daily dose of from 10 mg to 1000 mg, while an intravenous dose may only require from 1 mg to 500 mg. The total daily dose may be administered in single or divided doses and may, at the physician's discretion, fall outside of the typical range given herein.

These dosages are based on an average human subject having a weight of about 60 kg to 70 kg. The physician will readily be able to determine doses for subjects whose weight falls outside this range, such as infants and the elderly, and especially obese patients.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

The compounds with which the invention is concerned may be administered alone, or as part of a combination therapy with other drugs, especially those used for treatment of the diseases mentioned. In the treatment of obesity, a compound of the invention may be administered with an agent preventing central or peripheral food intake; modulating fat or protein metabolism or storage; preventing fat absorption; or increasing thermogenesis, In the treatment of other disorders related to obesity a compound of this invention could administered with agents used for treatment of diseases like metabolic syndrome; Type II diabetes; bulimia; cardiovascular disease including dyslipidemia, myocardial infarction, and hypertension; osteoarthritis; obesity-related cancers, In the treatment of mental disorders a compound of this invention could administered with agents used for treatment of diseases like depression, anxiety, mood disorders, abuse disorders, cognitive disorders, sleep disorders, psychosis, and dementia.

### Preparation of Compounds Useful to the Invention:

There are multiple synthetic routes for the preparation of the compounds (I) with which the present invention is concerned, but all rely on chemistry known to the synthetic organic chemist. Thus, compounds represented by Formula I can be synthesized according to procedures described in the literature and are well-known to one skilled in the art. Typical literature sources are “Advanced organic chemistry”, 4th Edition (Wiley), J March, “Comprehensive Organic Transformation”, 2nd Edition (Wiley), R. C. Larock, “Handbook of Heterocyclic Chemistry”, 2nd Edition (Pergamon), A. R. Katritzky), review articles such as found in “Synthesis”, “Acc. Chem. Res.”, “Chem. Rev”, or primary literature sources identified by standard literature searches online or from secondary sources such as “Chemical Abstracts” or “Beilstein”. Compounds of the invention can be synthesized by methods analogous to those exemplified in the Examples herein for certain representative compounds.

A compound of Formula (I) can for example be made by introducing the A₁-R₁ moiety to a suitable activated 4-pyrazole intermediate such as the bromomethyl derivative as outlined below:

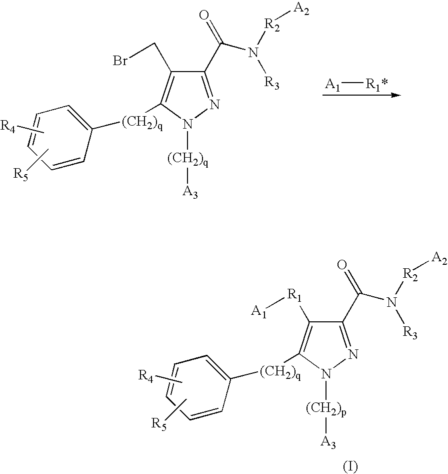

Thus, the A₁-R₁* moiety contains a nucleophilic oxygen, sulphur, nitrogen or carbon center and the remaining part could constitute the final substituent or constitute a protected version of the substituent: For example by reacting the bromo derivative with potassium cyanide, the nitrile obtained can subsequently be converted to a tetrazole moiety by reaction with azide or to a carboxylic acid by hydrolysis.

Alternatively, the compounds of Formula I may be obtained by introduction of the N(R₃)R₂-A₂ moiety to a corresponding carboxylic acid or a protected form of the depicted carboxyclic acid as outlined in the following scheme:

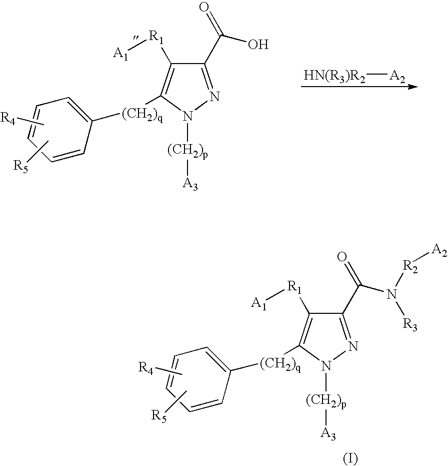

Thus, the HN(R₃)R₂-A₂ moiety contains a nucleophilic nitrogen center and the remaining part could constitute the final substituent or a protected version of the substituent. Thus, compounds of Formula I are either obtained directly or compounds (I) are obtained after standard conversions or removal of protecting groups. The carboxylic acids can be in activated forms, e.g. acid chlorides or active esters. Alternatively, the conversion can be made directly using suitable coupling reagents such as dicyclohexylcarbodiimide (DCC), and promoters such as 1-hydroxybenzotriazole.

Alternatively, the substituents R₄ and R₅ can be introduced at a final stage in the phenyl ring or into the A₃ moiety as indicated in the route below by reacting a suitable precursor such as a bromo compound with a suitable reagent such as zinc cyanide in the presence of a metal catalyst such as a palladium(0) complex.

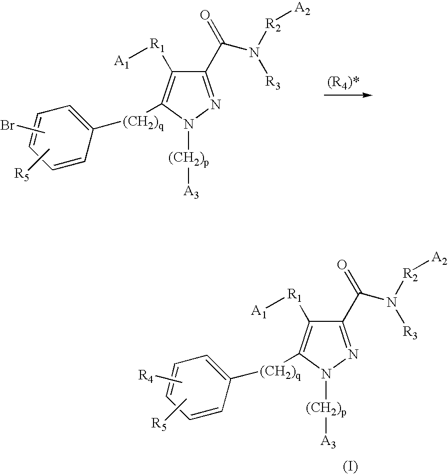

The conversion may also be made on an intermediate that can be converted to the compounds of Formula I or on a protected version of the intermediates. Analogously, substituents may also be introduced in the R₂ moiety at the final stage of the reaction sequence.

The experimental section contains examples of the different synthetic routes and the person skilled in the art may apply analogous routes using procedures found in the literature to make compounds represented by Formula I.

Embodiments of the invention are illustrated in the following Examples:

### Analysis:

APCI-LCMS analysis was obtained under standardised conditions either (A) or (B) as follows:
(A) Column; XTerra MS C18, 2.5□m, 4.6×20 mm. Flow: 1.5 ml/min. Gradient: Acetonitrile in 0.1% aqueous formic acid: 10% acetonitrile for 0.25 minutes, 10%-100% acetonitrile over 2.50 minutes then 100% acetonitrile for 1.75 minutes. Instrument: Thermo Finnigan Surveyor MSQ, 254 nm UV detection.
(B) Column; Gemini C18, 5 μm, 2.0×50 mm. Flow: 1.2 ml/min; Gradient: Acetonitrile in 0.1% aqueous trifluoroacetic acid: 10%-95% acetonitrile over 3.5 minutes then 95% acetonitrile for 1.0 minute. Instrument: Agilent 1100 HPLC/MSD system, 254 nm UV detection.

Data is quoted for all compounds as molecular ion and retention time (RT) using method (A) unless otherwise stated.

¹H NMR resonances were measured on a Bruker 400 MHz. spectrometer and chemical shifts are quoted for selected compounds in parts-per-million (ppm) downfield relative to tetramethylsilane as internal standard.

### Compounds of General Formula [1]

| | |
|---|---|
| | [1] |
| 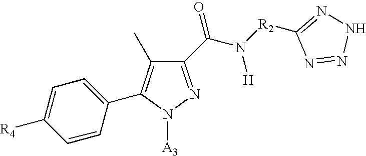 | |

| | | | | | Analysis |
|---|---|---|---|---|---|
| Compound | | | | | HPLC RT |
| Number | Compound Name | A₃ | R₂ | R₄ | MS (APCI+) |
| 1.01 | 5-(4-Chloro-phenyl)-1-(2,4-dichloro-phenyl)-4-methyl-1H-pyrazole-3-carboxylic acid [3-(1H-tetrazol-5-yl)-propyl]-amide | 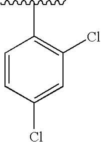 | —(CH₂)₃— | Cl | 2.79 min.491 (M + H)⁺ |
| 1.02 | 5-(4-Chloro-phenyl)-1-(2,4-dichloro-phenyl)-4-methyl-1H-pyrazole-3-carboxylic acid (2H-tetrazol-5-yl)-amide | 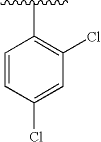 | Bond | Cl | 2.90 min.448 (M + H)⁺ |
| 1.03 | 5-(4-Chloro-phenyl)-1-(2,4-dichloro-phenyl)-4-methyl-1H-pyrazole-3-carboxylic acid [2-(2H-tetrazol-5-yl)-ethyl]-amide | 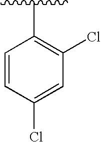 | —(CH₂)₂— | Cl | 2.75 min.476 (M + H)⁺ |
| 1.04 | 1,5-Bis-(4-chloro-phenyl)-4-methyl-1H-pyrazole-3-carboxylic acid [3-(2H-tetrazol-5-yl)-propyl]-amide | 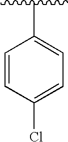 | —(CH₂)₃— | Cl | 2.74 min.456 (M + H)⁺ |
| 1.05 | 5-(4-Chloro-phenyl)-4-methyl-1-phenyl-1H-pyrazole-3-carboxylicacid [3-(2H-tetrazoi-5-yl)-propyl]-amide | 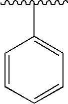 | —(CH₂)₃— | Cl | 2.58 min.422 (M + H)⁺ |
| 1.06 | 5-(4-Chloro-phenyl)-1-(2,4-dichloro-phenyl)-4-methyl-1H-pyrazole-3-carboxylic acid [5-(2H-tetrazol-5-yl)-pentyl]-amide | 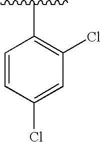 | —(CH₂)₅— | Cl | 2.89 min.518 (M + H)⁺ |
| 1.07 | 5-(4-Chloro-phenyl)-1-cyclohexyl-4-methyl-1H-pyrazole-3-carboxylicacid [3-(2H-tetrazol-5-yl)-propyl]-amide | 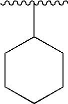 | —(CH₂)₃— | Cl | 2.79 min.428 (M + H)⁺ |
| 1.08 | 5-(4-Chloro-phenyl)-1-(2,4-dichloro-phenyl)-4-methyl-1H-pyrazole-3-carboxylic acid (2H-tetrazol-5-ylmethyl)-amide | 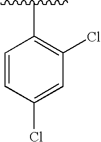 | —CH₂— | Cl | 2.76 min.462 (M + H)⁺ |
| 1.09 | 1-(2,4-Dichloro-phenyl)-4-methyl-5-(4-sulfamoyl-phenyl)1H-pyrazole-3-carboxylic acid [3-(2H-tetrazol-5-yl)-propyl]-amide | 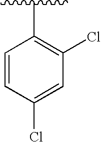 | —(CH₂)₃— | —SO₂NH₂ | 2.18 min.536 (M + H)⁺ |
| 1.10 | 1-(2,4-Dichloro-phenyl)-4-methyl-5-(4-propoxy-phenyl)-1H-pyrazole-3-carboxylic acid [3-(2H-tetrazol-5-yl)-propyl]-amide | 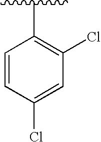 | —(CH₂)₃— | Propoxy | 2.88 min.515 (M + H)⁺ |
| 1.11 | 5-(4-Chloro-phenyl)-1-(2-chloro-4-propoxy-phenyl)-4-methyl-1H-pyrazole-3-carboxylic acid [3-(2H-tetrazol-5-yl)-propyl]-amide | 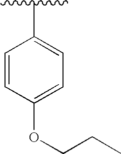 | —(CH₂)₃— | Cl | 2.76 min.480 (M + H)⁺ |
| 1.12 | 5-(4-Chloro-phenyl)-4-methyl-1-(3-sulfamoyl-phenyl)-1H-pyrazole-3-carboxylic acid [3-(2H-tetrazol-5-yl)-propyl]-amide | 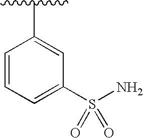 | —(CH₂)₃— | Cl | 2.54 min.501 (M + H)⁺ |

### Synthesis:

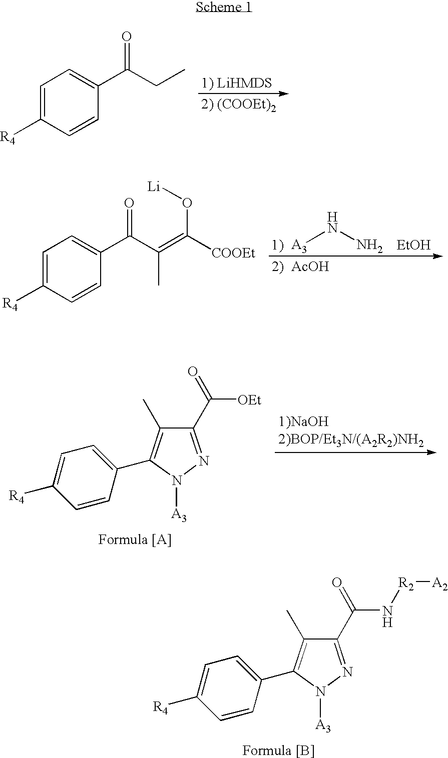

Amide intermediates of formula [B] were prepared as described in scheme 1 above. Ester derivatives of formula [A] were obtained by well known methods (Ruoxi et al., J. Med. Chem, 1999, 42, 769-776). These esters were hydrolysed with sodium hydroxide under conditions familiar to those skilled in the art and the resulting acids coupled with amines A₂R₂NH₂ according to the procedure of Seltzman et al., J. Chem. Soc., Chem. Commun., 1995, 1549-1550.

### Compound 1.01

Prepared according to the procedure outlined in scheme 2:

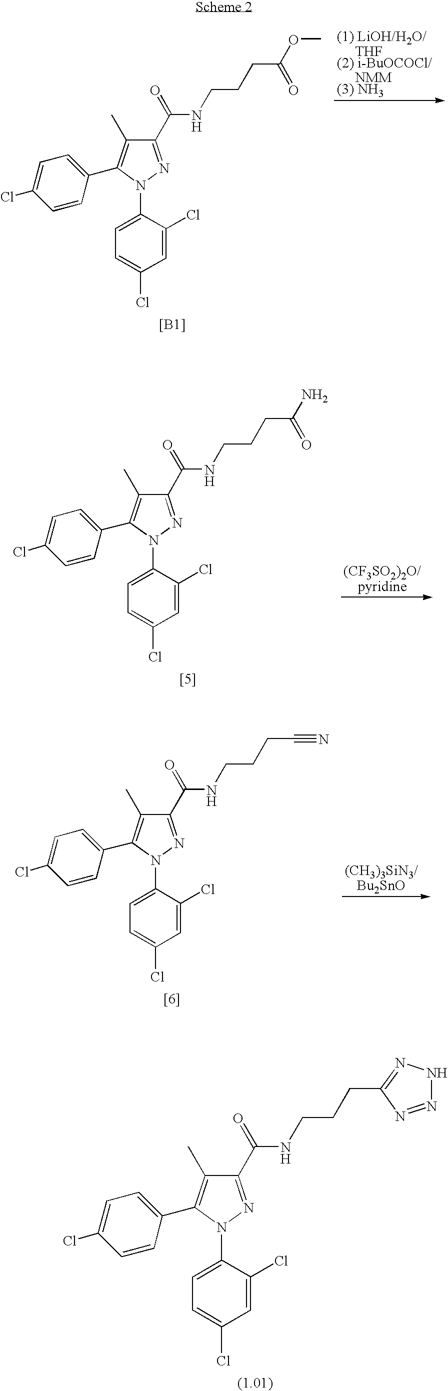

4-{[5-(4-Chloro-phenyl)-1-(2,4-dichloro-phenyl)-4-methyl-1H-pyrazole-3-carbonyl]-amino}-butyric acid methyl ester [α] was obtained according to the procedure outlined in scheme 1 using 4-amino-butyric acid methyl ester hydrochloride as amine A₂R₂NH₂.

[B1](0.63 g, 1.31 mmol) was hydrolysed, under conditions familiar to those skilled in the art with lithium hydroxide in tetrahydrofuran/water (1/1). To a stirred solution of the resulting acid in dichloromethane (20 ml) at 0° C., was added N-methylmorpholine (147 mg, 1.45 mmol) and isobutyl chloroformate (187 mg, 1.37 mmol). After 1 hour, aqueous ammonium hydroxide solution (4 mL) was added and the organic solvent was evaporated. The aqueous residue was treated with 3M hydrochloric acid and extracted with ethyl acetate. Evaporation of the organic phase gave 5-(4-chloro-phenyl)-1-(2,4-dichloro-phenyl)-4-methyl-1H-pyrazole-3-carboxylic acid (3-carbamoyl-propyl)-amide [5].

To a stirred solution of [5] in dichloromethane (30 ml) at room temperature was added trifluoroacetic anhydride (0.93 ml, 6.55 mmol) and pyridine (1.06 ml, 13.1 mmol). After 16 hours, the mixture was evaporated under reduced pressure. The residue was dissolved in ethanol and the solution heated at reflux for 30 minutes then evaporated. The residue was crystallised from ether/pentane to give 5-(4-chloro-phenyl)-1-(2,4-dichloro-phenyl)-4-methyl-1H-pyrazole-3-carboxylic acid (3-cyano-propyl)-amide [6] (0.56 g, 1.25 mmol).

Compound [6] (1.95 g, 4.34 mmol) was dissolved in degassed toluene (10 ml) and to the solution was added dibutyltin oxide (1.08 g, 4.34 mmol) and azidotrimethylsilane (0.75 g, 6.51 mmol). The mixture was heated at 100° C. in a sealed tube for 16 hours then partitioned between water and ether. The organic phase was dried over sodium sulphate, filtered and evaporated. The residue was purified by chromatography over silica, eluting with an ethyl acetate/ethanol gradient followed by recrystallisation from a mixture of isopropanol and isopropyl ether to give the title compound [1.01] (1.25 g, 2.55 mmol, 59%).

¹H NMR (DMSO-D₆): δ 1.97 (2H, quin.), 2.29 (3H, s), 2.92 (2H, t), 3.34 (2H, t), 7.25 (2H, d), 7.48 (2H, d), 7.61 (1H, t), 7.76-7.80 (2H, m), 8.43 (1H, dd), 16.02 (1H, s, br).

Compounds 1.04, 1.05, 1.07, 1.09, 1.10, 1.11 and 1.12 were synthesized as described for compound 1.1. Whereas compound 1.06 was obtained by an analogous procedure but using 6-amino-hexanoic acid methyl ester as amine A₂R₂NH₂. In these examples, the final step (tetrazole formation) could either be according to the procedure described for compound 1.1 or other methods known to those skilled in the art e.g. sodium azide and triethylamine hydrochloride in toluene (Koguro et al., 1998, Synthesis, 910-914).

Compounds 1.02, 1.03 and 1.08 were obtained by using cyanamide, 3-amino-propionitrile and 2-aminoacetonitrile respectively as amine (A₂R₂)NH₂ (scheme 1), followed directly by tetrazole formation as described above.

### Compounds of General Formula [2]

| | |
|---|---|
| | [2] |
| 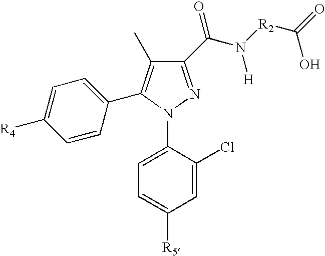 | |

| | | | | | Analysis |
|---|---|---|---|---|---|
| Compound | | | | | HPLC RT |
| Number | Compound Name | R₂ | R₄ | R_{5′} | MS (APCI+) |
| 2.01 | 4-{[5-(4-Chloro-phenyl)-1-(2,4- | —(CH₂)₃— | Cl | Cl | 2.85 min. |
| | dichloro-phenyl)-4-methyl-1H- | | | | 466 (M + H)⁺ |
| | pyrazole-3-carbonyl]-amino}-butyric | | | | |
| | acid | | | | |
| 2.02 | (4-{[5-(4-Chloro-phenyl)-1-(2,4-dichloro-phenyl)-4-methyl-1H-pyrazole-3-carbonyl]-amino}-phenyl)-acetic acid | 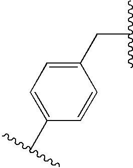 | Cl | Cl | 3.15 min.514 (M + H)⁺ |
| 2.03 | 4-{[1-(2-Chloro-phenyl)-5-(4- | —(CH₂)₃— | OH | H | 2.10 min. |
| | hydroxy-phenyl)-4-methyl-1H- | | | | 414 (M + H)⁺ |
| | pyrazole-3-carbonyl]-amino}-butyric | | | | |
| | acid | | | | |
| 2.04 | [4-({1-(2-Chloro-phenyl)-4-methyl-5-[4-(2-methyl-2H-tetrazol-5-yl)-phenyl]-1H-pyrazole-3-carbonyl}-amino)-phenyl]-acetic acid | 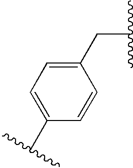 | 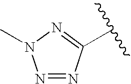 | H | 2.67 min.528 (M + H)⁺ |
| 2.05 | 4-({1-(2-Chloro-phenyl)-4-methyl-5-[4-(2-methyl-2H-tetrazol-5-yl)-phenyl]-1H-pyrazole-3-carbonyl)-amino)-butyric acid | —(CH₂)₃— | 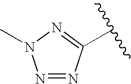 | H | 2.36 min.480 (M + H)⁺ |
| 2.06 | 4-{[1-(2-Chloro-phenyl)-4-methyl-5- | —(CH₂)₃— | —SO₂NH₂ | H | 2.04 min. |
| | (4-sulfamoyl-phenyl)-1H-pyrazole-3- | | | | 477 (M + H)⁺ |
| | carbonyl]-amino}-butyric acid | | | | |

### Synthesis:

Compounds 2.01 and 2.02 were obtained by hydrolysis with potassium hydroxide in methanol, of their corresponding esters under conditions familiar to those skilled in the art. The esters were prepared as compounds of formula [B] using the procedure outlined in scheme 1 wherein A₂R₂NH₂ was 4-amino-butyric acid methyl ester or (4-amino-phenyl)-acetic acid ethyl ester respectively.

### Compound 2.03

Prepared according to the procedure outlined in scheme 3:

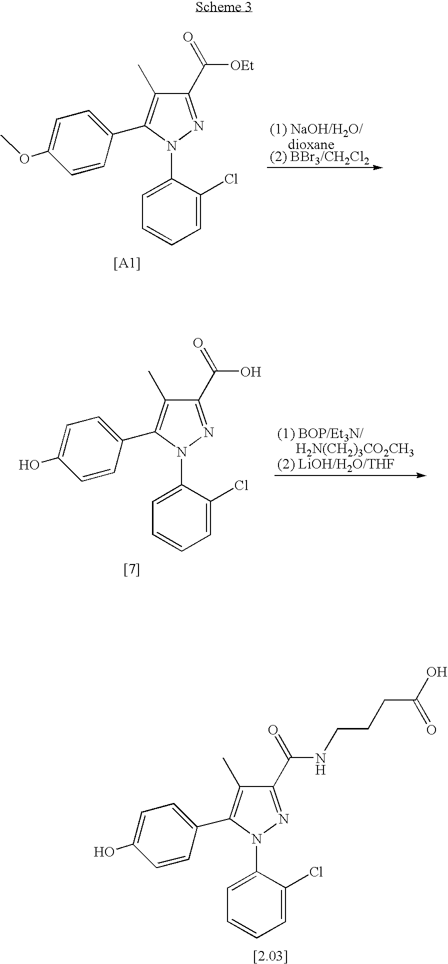

1-(2-Chloro-phenyl)-5-(4-methoxy-phenyl)-4-methyl-1H-pyrazole-3-carboxylic acid ethyl ester

[A1] was prepared according to the procedure outlined in scheme 1. [A1] was hydrolysed with sodium hydroxide in 1,4-dioxane/water (2:1) under conditions familiar to those skilled in the art. To a stirred solution of the resulting acid (0.30 g, 0.88 mmol) in dichloromethane (10 ml) at 0° C. was added boron tribromide (2.63 mmol). The solution was stirred at room temperature for 16 hours and then neutralized with sodium bicarbonate. The organic phase was filtered and evaporated to give crude 1-(2-chloro-phenyl)-5-(4-hydroxy-phenyl)-4-methyl-1H-pyrazole-3-carboxylic acid [7] as a brown solid. This acid was coupled with 4-amino-butyric acid methyl ester using similar conditions to those outlined in scheme 1. The resulting ester was hydrolysed with lithium hydroxide in tetrahydrofuran/water (1/1) under conditions familiar to those skilled in the art to give the title compound [2.03] (0.04 g, 0.10 mmol, 11%).

¹H NMR (CDCl₃): δ 1.85-1.95 (2H, quin), 2.33 (3H, s), 2.41 (2Ht), 2.78 (3H, s, br), 3.45 (2H, t), 6.71 (2h, d), 6.95 (2H, d), 7.24-7.34, (3H, m), 7.39 (1H, d).

### Compound 2.04

Prepared according to the procedure outlined in scheme 4:

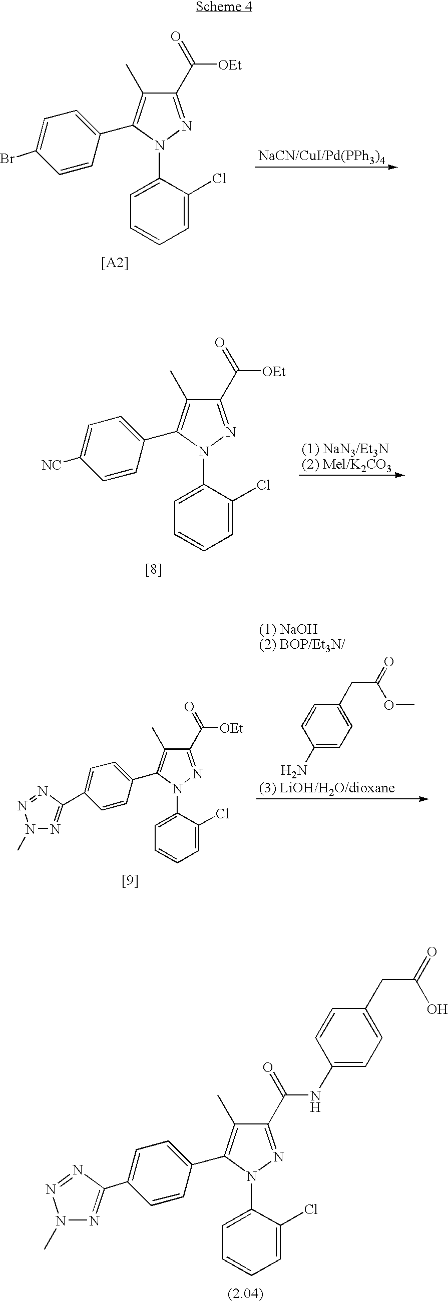

5-(4-Bromo-phenyl)-1-(2-chloro-phenyl)-4-methyl-1H-pyrazole-3-carboxylic acid ethyl ester [A2] was obtained according the procedure outlined in scheme 1.

A solution of [A2] (3 g, 7.15 mmol), tetrakis(triphenylphosphine)palladium(0) (0.825 g, 0.715 mmol), sodium cyanide (1.4 g, 28.6 mmol) and copper(I)iodide (272 mg, 1.43 mmol) in tetrahydrofuran (15 ml) was purged with argon and heated at 60° C. in a sealed tube for 16 hours. The mixture was filtered and the solution evaporated. The residue was purified by chromatography over silica, eluting with ethyl acetate/cyclohexane (1/4) to give 1-(2-chloro-phenyl)-5-(4-cyano-phenyl)-4-methyl-1H-pyrazole-3-carboxylic acid ethyl ester [8] (0.60 g).

A mixture of [8] (1.8 g, 4.9 mmol), sodium azide (0.955 g, 14.7 mmol) and triethylamine hydrochloride (2.0 g, 14.7 mmol) in toluene (10 ml) was heated at 80° C. for 15 hours. Solvent was evaporated and the residue redissolved in acetonitrile (10 ml). Potassium carbonate (0.64 g, 0.46 mmol) and iodomethane (0.58 ml, 9.2 mmol) were added and the mixture stirred at 20° C. for 3 hours. The mixture was filtered through a pad of silica, washing with ethyl acetate and the filtrate evaporated. The residue was purified by chromatography over silica, eluting with a mixture of ethyl acetate and cyclohexane to give the major regioisomer 1-(2-chloro-phenyl)-4-methyl-5-[4-(2-methyl-2H-tetrazol-5-yl)-phenyl]-1H-pyrazole-3-carboxylic acid ethyl ester [9] (1.25 g, 3.0 mmol).

Compound [9] was hydrolysed with sodium hydroxide in 1,4-dioxane/water (5/3) under conditions familiar to those skilled in the art. The resulting acid (0.10 g, 0.25 mmol) was coupled with (4-amino-phenyl)-acetic acid methyl ester using similar conditions to those outlined in scheme 1. Hydrolysis of the ester with lithium hydroxide in 1,4-dioxane/water (1/1) under conditions familiar to those skilled in the art gave the title compound [2.04] (0.076 g, 0.14 mmol, 56%) as a white solid.

¹H NMR (CDCl₃): δ2.48 (3H, s), 2.95 (1H, s, br), 3.58 (2H, s), 4.39 (3H, s), 7.23-7.44 (8H, m), 7.64 (2H, d), 8.04 (2H, d), 8.94 (1H, s, br).

Compound 2.05 was obtained as described for compound 2.04, using 4-amino-butyric acid methyl ester in place of (4-amino-phenyl)-acetic acid methyl ester.

### Compound 2.06

Prepared according to the procedure outlined in scheme 5:

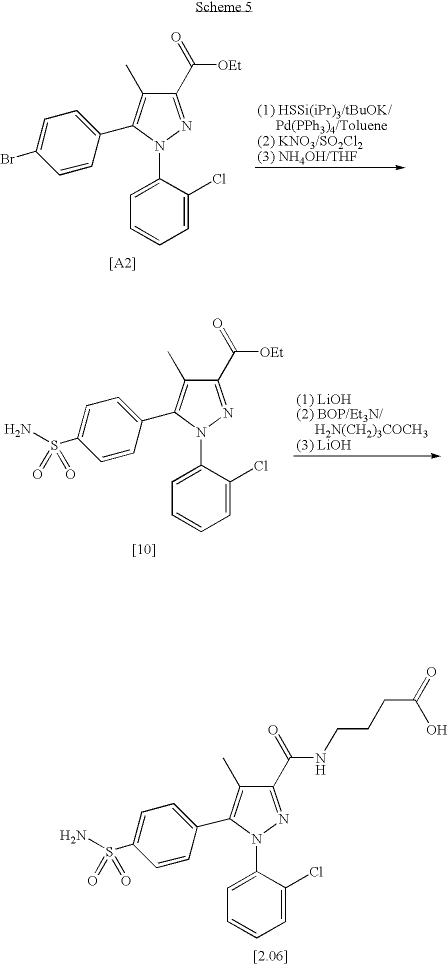

The transformation of bromo to sulfonamide was according to a method described in the literature (Gareau et al., Tetrahedron Letters, 2003, 44, 7821-7824):

A solution of intermediate [A2] (1.00 g, 2.38 mmol), potassium tert.-butoxide (0.80 g, 7.15 mmol), tetrakis(triphenylphosphine)palladium(0) (0.28 g, 0.238 mmol) and triisopropylsilanethiol (0.91 g, 4.76 mmol) in degassed toluene was stirred at 100° C. for 3 hours. The solution was evaporated and the residue purified by chromatography over silica gel, eluting with ethyl acetate/cyclohexane (1/9). To a solution of the resulting product in acetonitrile (20 ml) at 0° C., was added potassium nitrate (0.25 g, 2.48 mmol) and sulphuryl chloride (0.34 g, 2.49 mmol). The mixture was stirred for 1 hour at 0° C. then neutralized with aqueous sodium carbonate solution and extracted with ether. The organic phase was washed with brine, dried over sodium sulphate, filtered and evaporated. The residue was dissolved in THF (10 ml) and aqueous ammonium hydroxide solution (3 ml) was added. The solution was stirred for 15 minutes then evaporated. The residue was partitioned between 3M hydrochloric acid and ethyl acetate. The organic phase was washed with 10% sodium hydroxide solution, dried over sodium sulphate, filtered and evaporated. The residue was purified by chromatography over silica, eluting with ethyl acetate to give 1-(2-chloro-phenyl)-4-methyl-5-(4-sulfamoyl-phenyl)-1H-pyrazole-3-carboxylic acid ethyl ester [10] (0.195 g, 0.46 mmol).

Hydrolysis of [10] (0.196 g, 0.46 mmol) with lithium hydroxide in tetrahydrofuran/water under conditions familiar to those skilled in the art followed by coupling with 4-amino-butyric acid methyl ester then hydrolysis under similar conditions gave the title compound [2.06] (0.097 g, 0.20 mmol, 43%) as a white solid.

¹H NMR (DMSO-D₆): 1.68-1.78 (2H, m), 2.18-2.32 (5H, m), 3.18-3.28 (2H, m), 7.37-7.57 (7H, m), 7.68-7.82 (3H, m), 8.32 (1H, t, br).

Compounds of General Formula 3

| | |
|---|---|
| | [3] |
| 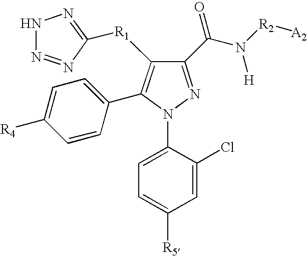 | |

| | | | | | | Analysis |
|---|---|---|---|---|---|---|
| Compound | | | | | | HPLC RT |
| Number | Compound Name | R₁ | —R₂—A₂ | R₄ | R_{5′} | MS (APCI+) |
| 3.01 | 5-(4-Chloro-phenyl)-1-(2,4-dichloro-phenyl)-4-(2H-tetrazol-5-ylmethyl)-1H-pyrazole-3-carboxylic acidpiperidin-1-ylamide | —CH₂— | 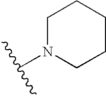 | Cl | Cl | 2.76 min.531 (M + H)⁺ |
| 3.02 | 5-(4-Chloro-phenyl)-1-(2,4-dichloro-phenyl)-4-(2H-tetrazol-5-ylmethyl)-1H-pyrazole-3-carboxylic acidmorpholin-4-ylamide | —CH₂— | 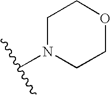 | Cl | Cl | 2.66 min.533 (M + H)⁺ |
| 3.03 | 5-(4-Chloro-phenyl)-1-(2,4- | —CH₂— | —CH₂CF₃ | Cl | Cl | 2.96 min. |
| | dichloro-phenyl)-4-(2H- | | | | | 530 (M + H)⁺ |
| | tetrazol-5-ylmethyl)-1H- | | | | | |
| | pyrazole-3-carboxylic acid | | | | | |
| | (2,2,2-trifluoro-ethyl)-amide | | | | | |
| 3.04 | 5-(4-Chloro-phenyl)-1-(2,4-dichloro-phenyl)-4-(2H-tetrazol-5-ylmethyl)-1H-pyrazole-3-carboxylic acidcyclopropylmethyl-amide | —CH₂— | 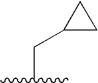 | Cl | Cl | 3.01 min.502 (M + H)⁺ |
| 3.05 | 5-(4-Chloro-phenyl)-1-(2,4-dichloro-phenyl)-4-(2H-tetrazol-5-ylmethyl)-1H-pyrazole-3-carboxylic acid(4-fluoro-phenyl)-amide | —CH₂— | 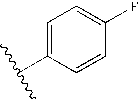 | Cl | Cl | 3.18 min.542 (M + H)⁺ |
| 3.06 | 5-(4-Chloro-phenyl)-1-(2,4-dichloro-phenyl)-4-(2H-tetrazol-5-ylmethyl)-1H-pyrazole-3-carboxylic acid 4-fluoro-benzylamide | —CH₂— | 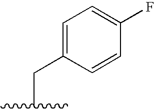 | Cl | Cl | 3.14 min.556 (M + H)⁺ |
| 3.07 | 5-(4-Chloro-phenyl)-1-(2,4-dichloro-phenyl)-4-(2H-tetrazol-5-ylmethyl)-1H-pyrazole-3-carboxylic acidpyrimidin-5-ylamide | —CH₂— | 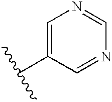 | Cl | Cl | 2.83 min.526 (M + H)⁺ |
| 3.08 | 5-(4-Chloro-phenyl)-1-(2,4-dichloro-phenyl)-4-(2H-tetrazol-5-ylmethyl)-1H-pyrazole-3-carboxylic acid(4-sulfamoyl-phenyl)-amide | —CH₂— | 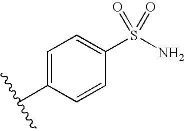 | Cl | Cl | 2.79 min.603 (M + H)⁺ |
| 3.09 | 5-(4-Chloro-phenyl)-1-(2,4-dichloro-phenyl)-4-(2H-tetrazol-5-ylmethyl)-1H-pyrazole-3-carboxylic acidcyclopentylamide | —CH₂— | 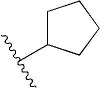 | Cl | Cl | 3.11 min.516 (M + H)⁺ |
| 3.10 | 5-(4-Chloro-phenyl)-1-(2,4-dichloro-phenyl)-4-(1H-tetrazol-5-ylmethyl)-1H-pyrazole-3-carboxylic acid(3-fluoro-phenyl)-amide | —CH₂— | 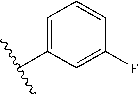 | Cl | Cl | 3.21 min.542 (M + H)⁺ |
| 3.11 | 5-(4-Chloro-phenyl)-1-(2,4-dichloro-phenyl)-4-(1H-tetrazol-5-ylmethyl)-1H-pyrazole-3-carboxylic acidazepan-1-ylamide | —CH₂— | 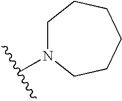 | Cl | Cl | 3.05 min.545 (M + H)⁺ |
| 3.12 | 5-(4-Chloro-phenyl)-1-(2,4-dichloro-phenyl)-4-(1H-tetrazol-5-ylmethyl)-1H-pyrazole-3-carboxylic acidpyrimidin-4-ylamide | —CH₂— | 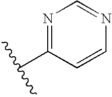 | Cl | Cl | 2.86 min.526 (M + H)⁺ |
| 3.13 | 5-(4-Chloro-phenyl)-1-(2,4-dichloro-phenyl)-4-(2H-tetrazol-5-ylmethyl)-1H-pyrazole-3-carboxylic acid(3-sulfamoyl-phenyl)-amide | —CH₂— | 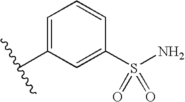 | Cl | Cl | 2.84 min.603 (M + H)⁺ |
| 3.14 | 5-(4-Chloro-phenyl)-1-(2,4-dichloro-phenyl)-4-(2H-tetrazol-5-ylmethyl)-1H-pyrazole-3-carboxylic acid(4-methoxy-phenyl)-amide | —CH₂— | 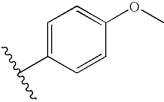 | Cl | Cl | 3.16 min.554 (M + H)⁺ |
| 3.15 | 5-(4-Chloro-phenyl)-1-(2,4-dichloro-phenyl)-4-(2H-tetrazol-5-ylmethyl)-1H-pyrazole-3-carboxylic acid[2-(4-fluoro-phenyl)-ethyl]-amide | —CH₂— | 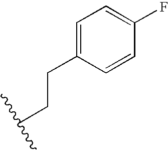 | Cl | Cl | 3.24 min.570 (M + H)⁺ |
| 3.16 | 5-(4-Chloro-phenyl)-1-(2,4-dichloro-phenyl)-4-(2H-tetrazol-5-ylmethyl)-1H-pyrazole-3-carboxylic acid 4-methoxy-benzylamide | —CH₂— | 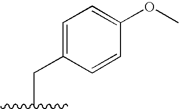 | Cl | Cl | 3.11 min.568 (M + H)⁺ |
| 3.17 | 5-(4-Chloro-phenyl)-1-(2,4-dichloro-phenyl)-4-(2H-tetrazol-5-ylmethyl)-1H-pyrazole-3-carboxylic acid[3-(2-oxo-pyrrolidin-1-yl)-propyl]-amide | —CH₂— | 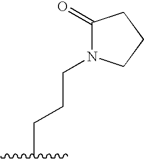 | Cl | Cl | 2.70 min.573 (M + H)⁺ |
| 3.18 | 5-(4-Chloro-phenyl)-1-(2,4-dichloro-phenyl)-4-(2H-tetrazol-5-ylmethyl)-1H-pyrazoie-3-carboxyiic acidpyridin-4-ylamide | —CH₂— | 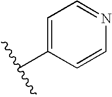 | Cl | Cl | 2.28 min.525 (M + H)⁺ |
| 3.19 | 5-(4-Chloro-phenyl)-1-(2,4-dichloro-phenyl)-4-(2H-tetrazol-5-ylmethyl)-1H-pyrazole-3-carboxylic acidpyridin-3-ylamide | —CH₂— | 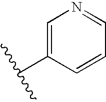 | Cl | Cl | 2.47 min.525 (M + H)⁺ |
| 3.20 | 1-(2,4-Dichloro-phenyl)-4-(2H-tetrazol-5-ylmethyl)-5-p-tolyl-1H-pyrazole-3-carboxylic acidcyclohexylamide | —CH₂— | 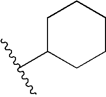 | —CH₃ | Cl | 3.10 min.510 (M + H)⁺ |
| 3.21 | 5-(4-Chloro-phenyl)-1-(2,4- | —CH₂— | —(CH₂)₃CF₃ | Cl | Cl | 3.12 min. |
| | dichloro-phenyl)-4-(2H- | | | | | 558 (M + H)⁺ |
| | tetrazol-5-ylmethyl)-1H- | | | | | |
| | pyrazole-3-carboxylic acid | | | | | |
| | (4,4,4-trifluoro-butyl)-amide | | | | | |
| 3.22 | 5-(4-Chloro-phenyl)-1-(2,4-dichloro-phenyl)-4-(2H-tetrazol-5-ylmethyl)-1H-pyrazole-3-carboxylic acid(4-chloro-phenyl)-amide | —CH₂— | 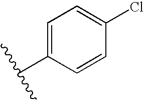 | Cl | Cl | 3.29 min.560 (M + H)⁺ |
| 3.23 | 5-(4-Chloro-phenyl)-1-(2,4- | —CH₂— | —(CH₂)₃SO₂NH₂ | Cl | Cl | 2.64 min. |
| | dichloro-phenyl)-4-(2H- | | | | | 569 (M + H)⁺ |
| | tetrazol-5-ylmethyl)-1H- | | | | | |
| | pyrazole-3-carboxylic acid | | | | | |
| | (3-sulfamoyl-propyl)-amide | | | | | |
| 3.24 | 5-(4-Chloro-phenyl)-1-(2,4-dichloro-phenyl)-4-(2H-tetrazol-5-ylmethyl)-1H-pyrazoie-3-carboxylic acid 4-sulfamoyl-benzylamide | —CH₂— | 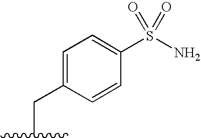 | Cl | Cl | 2.81 min.617 (M + H)⁺ |
| 3.25 | 5-(4-Chloro-phenyl)-1-(2,4-dichloro-phenyl)-4-(2H-tetrazol-5-ylmethyl)-1H-pyrazole-3-carboxylic acid(3-[1,2,3]triazol-1-yl-propyl)-amide | —CH₂— | 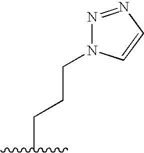 | Cl | Cl | 2.70 min.557 (M + H)⁺ |
| 3.26 | 5-(4-Chloro-phenyl)-1-(2,4-dichloro-phenyl)-4-(2H-tetrazol-5-ylmethyl)-1H-pyrazole-3-carboxylic acidpyrrolidin-1-ylamide | —CH₂— | 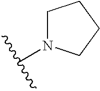 | Cl | Cl | 2.71 min.517 (M + H)⁺ |
| 3.27 | 5-(4-Chloro-phenyl)-1-(2,4-dichloro-phenyl)-4-(2H-tetrazol-5-ylmethyl)-1H-pyrazole-3-carboxylic acid(pyridin-4-ylmethyl)-amide | —CH₂— | 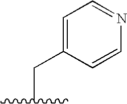 | Cl | Cl | 2.17 min.539 (M + H)⁺ |
| 3.28 | 5-(4-Chloro-phenyl)-1-(2,4-dichloro-phenyl)-4-(2H-tetrazol-5-ylmethyl)-1H-pyrazole-3-carboxylic acidN′-(4-fluoro-phenyl)-hydrazide | —CH₂— | 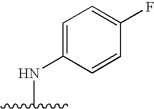 | Cl | Cl | 3.02 min.557 (M + H)⁺ |
| 3.29 | 1-(2,4-Dichloro-phenyl)-4-(2H-tetrazol-5-ylmethyl)-5-p-tolyl-1H-pyrazole-3-carboxylic acid pyridin-3-ylamide | —CH₂— | 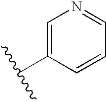 | —CH₃ | Cl | 2.38 min.505 (M + H)⁺ |
| 3.30 | 5-(4-Chloro-phenyl)-1-(2,4-dichloro-phenyl)-4-(2H-tetrazol-5-ylmethyl)-1H-pyrazole-3-carboxylic acid 4-carbamoyl-benzylamide | —CH₂— | 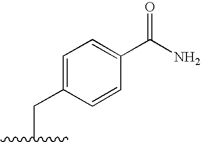 | Cl | Cl | 2.71 min.581 (M + H)⁺ |
| 3.31 | 5-(4-Cyano-phenyl)-1-(2,4-dichloro-phenyl)-4-(2H-tetrazol-5-ylmethyl)-1H-pyrazole-3-carboxylic acid 4-fluoro-benzylamide | —CH₂— | 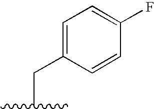 | —CN | H | 2.77 min.513 (M + H)⁺ |
| 3.32 | 1-(2,4-Dichloro-phenyl)-4-(2H-tetrazol-5-ylmethyl)-5-p-tolyl-1H-pyrazole-3-carboxylic acid pyridin-4-ylamide | —CH₂— | 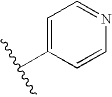 | —CH₃ | Cl | 2.19 min.505 (M + H)⁺ |
| 3.33 | 1-(2,4-Dichloro-phenyl)-4-(2H-tetrazol-5-ylmethyl)-5-p-tolyl-1H-pyrazole-3-carboxylic acid (1-oxy-pyridin-4-yl)-amide | —CH₂— | 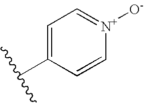 | —CH₃ | Cl | 2.47 min.521 (M + H)⁺ |
| 3.34 | 1-(2,4-Dichloro-phenyl)-4-(2H-tetrazol-5-ylmethyl)-5-p-tolyl-1H-pyrazole-3-carboxylic acid (pyridin-2-ylmethyl)-amide | —CH₂— | 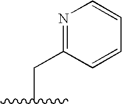 | —CH₃ | Cl | 2.50 min.519 (M + H)⁺ |
| 3.35 | 1-(2,4-Dichloro-phenyl)-4-(2H-tetrazol-5-ylmethyl)-5-p-tolyl-1H-pyrazole-3-carboxylic acid pyridin-2-ylamide | —CH₂— | 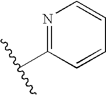 | —CH₃ | Cl | 2.99 min.505 (M + H)⁺ |
| 3.36 | 1-(2,4-Dichloro-phenyl)-4-(2H-tetrazol-5-ylmethyl)-5-p-tolyl-1H-pyrazole-3-carboxylic acid 4-sulfamoyl-benzylamide | —CH₂— | 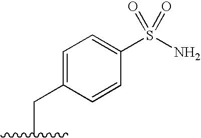 | —CH₃ | Cl | 2.75 min.597 (M + H)⁺ |
| 3.37 | 5-(4-Chloro-phenyl)-1-(2,4-dichloro-phenyl)-4-(2H-tetrazol-5-ylmethyl)-1H-pyrazole-3-carboxylic acid 4-cyano-benzylamide | —CH₂— | 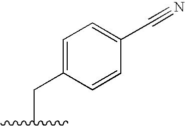 | Cl | Cl | 3.04 min.563 (M + H)⁺ |
| 3.38 | 5-(4-Chloro-phenyl)-1-(2,4-dichloro-phenyl)-4-(2H-tetrazol-5-yl)-1H-pyrazole-3-carboxylic acid4-methoxy-benzylamide | bond | 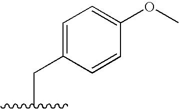 | Cl | Cl | 3.33 min.554 (M + H)⁺ |

### Synthesis:

Compounds of formula [D] were prepared according to the procedure outlined in scheme 6:

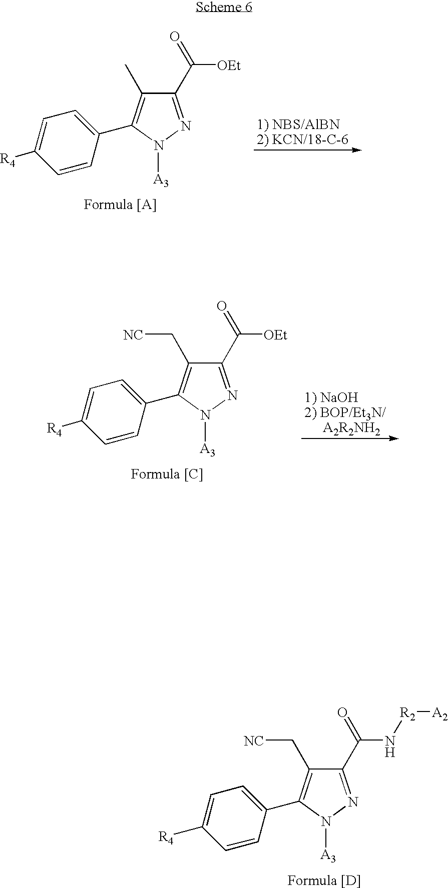

Compounds of formula [A] (prepared as outlined in scheme 1) were brominated by treatment with N-bromosuccinimide (1.1 eq.) and 1,1′-azobis(isobutyronitrile) (0.01 eq.) in carbon tetrachloride as described in US 2004/0192667. The resulting bromo compounds were not purified but treated directly with potassium cyanide (2 eq.) and 18-crown-6 ether (0.4 eq.) in acetonitrile at reflux for 15 hours. The reaction mixtures were partitioned between 1M sodium hydroxide solution and ethyl acetate. The organic phases were dried over sodium sulphate, filtered and evaporated. The residues were purified by chromatography over silica, eluting with ethyl acetate/cyclohexane mixtures to give compounds of formula [C].

Alternatively, the bromo compounds could be treated with sodium cyanide in a mixture of ethanol and water at reflux, which gave directly the hydrolysed derivatives of formula [C] esters.

Compounds of formula [C] were converted to compounds of formula [D] by well known methods using the appropriate amine A₂R₂NH₂, as previously outlined in scheme 1.

### Compound 3.01

Prepared according to the procedure outlined in scheme 7:

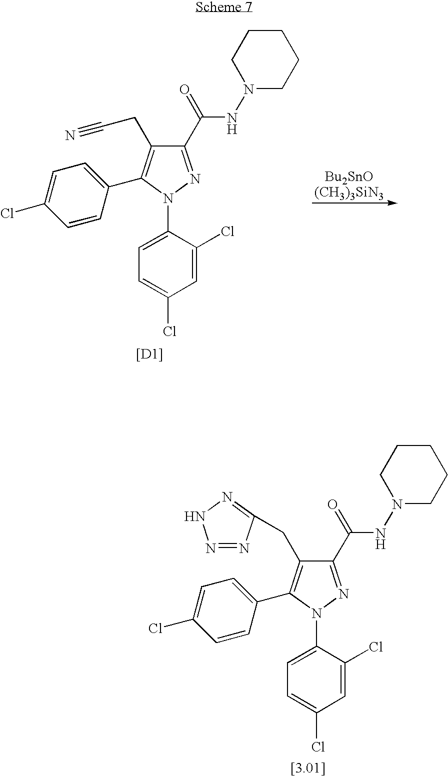

5-(4-Chloro-phenyl)-4-cyanomethyl-1-(2,4-dichloro-phenyl)-1H-pyrazole-3-carboxylic acid piperidin-1-ylamide [D1] was obtained according to the procedure outlined in scheme 6.

Dibutyltin oxide (0.083 g, 0.33 mmol) and azidotrimethylsilane (0.1 ml, 0.5 mmol) were added to a stirred solution of [D1] (0.16 g, 0.33 mmol) in toluene (5 ml) at 20° C. The solution was heated to 100° C. in a sealed tube for 2 hours. The reaction mixture was evaporated and the residue purified by chromatography over silica, eluting with a cyclohexane/ethyl acetate/acetic acid gradient to give the title compound [3.01] (0.091 g, 0.17 mmol, 52%) as a white solid.

¹H NMR (CDCl₃): δ0.87-0.91 (2H, m), 1.77-1.83 (2H, m), 2.85-2.93 (4H, m), 4.31 (2H, s), 7.21 (1H, d), 7.28-7.32 (3H, m), 7.41 (2H, d), 7.48 (1H, d), 7.90 (1H, s, br).

Compounds 3.2-3.37 were prepared in a similar manner to compound 3.01 from the appropriate compound of formula [D].

### Compound 3.38

Prepared according to the procedure outlined in scheme 8:

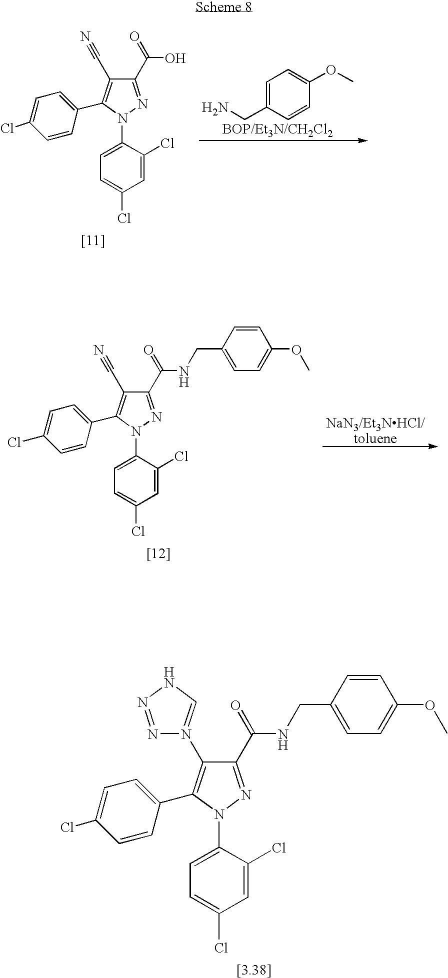

5-(4-Chlorophenyl)-4-cyano-1-(2,4-dichlorophenyl)-1H-pyrazole-3-carboxylic acid [1,1] (WO/2005000820) was coupled with 4-methoxybenzylamine according to the procedure previously outlined in scheme 1 to give 5-(4-chloro-phenyl)-4-cyano-1-(2,4-dichloro-phenyl)-1H-pyrazole-3-carboxylic acid 4-methoxy-benzylamide [12].

A suspension of compound [12] (0.10 g, 0.195 mmol), sodium azide 0.051 g, 0.784 mmol) and triethylamine hydrochloride (0.108 g, 0.784 mmol) in toluene (5 ml) was heated at 85° C. for 16 hours. The mixture was purified directly by chromatography over silica, eluting with ethyl acetate/cyclohexane (1:3). The resulting product was further purified by chromatography over silica, eluting with dichloromethane/ethyl acetate (95/5 then 7/3) to give the title compound [3.38] (0.016 g, 0.029 mmol, 15%) as a white solid.

¹H NMR (CDCl₃): δ 3.83 (3H, s), 4.65 (2H, d), 6.92 (2H, d), 7.28-7.34 (8H, m), 7.48 (1H, d), 7.72 (1H, t), 16.23 (1H, s, br).

### Compounds of General Formula [4]

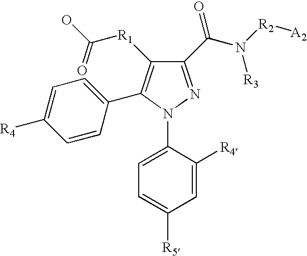

| | | | | | | | | Analysis |
|---|---|---|---|---|---|---|---|---|
| Compound | | | | | | | | HPLC RT |
| Number | Compound Name | R₁ | —R₂—A₂ | R₃ | R₄ | R_{4′} | R_{5′} | MS (APCI+) |
| 4.01 | [5-(4-Chloro-phenyl)-3-yclohexylcarbamoyl-1-(2,4-dichloro-phenyl)-1H-pyrazol-4-ylmethoxy]-acetic acid | —CH₂OCH₂— | 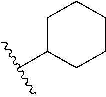 | H | Cl | Cl | Cl | 2.66 min.536 (M + H)⁺ |
| 4.02 | [5-(4-Chloro-phenyl)-1-(2,4-dichloro-phenyl)-3-(4-fluoro-phenylcarbamoyl)-1H-pyrazol-4-yl]-aceticacid | —CH₂— | 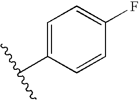 | H | Cl | Cl | Cl | 3.26 min.518 (M + H)⁺ |
| 4.03 | [1-(2-Chloro-phenyl)-5-(4-chloro-phenyl)-3-(4-fluoro-benzylcarbamoyl)-1H-pyrazol-4-yl]-aceticacid | —CH₂— | 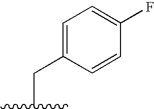 | H | Cl | Cl | H | 3.03 min.498 (M + H)⁺ |
| 4.04 | [5-(4-Chloro-phenyl)-1-(2-chloro-phenyl)-3-(4-cyano-benzylcarbamoyl)-1H-pyrazol-4-yl]-aceticacid | —CH₂— | 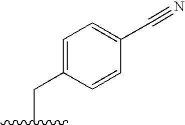 | H | Cl | Cl | H | 3.15 min.505 (M + H)⁺ |
| 4.05 | [5-(4-Chloro-phenyl)-1-l(2,4-dichloro-phenyl)-3-(4-cyano-benzylcarbamoyl)-1H-pyrazol-4-yl]-aceticacid | —CH₂— | 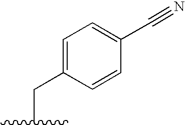 | H | Cl | Cl | Cl | 3.06 min.539 (M + H)⁺ |
| 4.06 | [5-(4-Chloro-phenyl)-1-(2,4-dichloro-phenyl)-3-(3-cyano-phenylcarbamoyl)-1H-pyrazol-4-yl]-aceticacid | —CH₂— | 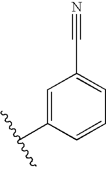 | H | Cl | Cl | Cl | 3.18 min.525 (M + H)⁺ |
| 4.07 | [5-(4-Chloro-phenyl)-1-(2,4-dichloro-phenyl)-3-(4-cyano-phenylcarbamoyl)-1H-pyrazol-4-yl]-aceticacid | —CH₂— | 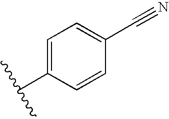 | H | Cl | Cl | Cl | 3.20 min.525 (M + H)⁺ |
| 4.08 | [5-(4-Chloro-phenyl)-1-(2,4-dichloro-phenyl)-3-(3-cyano-4-fluoro-benzylcarbamoyl)-1H-pyrazol-4-yl]-aceticacid | —CH₂— | 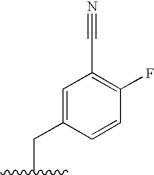 | H | Cl | Cl | Cl | 3.12 min.557 (M + H)⁺ |
| 4.09 | [5-(4-Chloro-phenyl)-1-(2-chloro-phenyl)-3-(4-cyano-2-fluoro-benzylcarbamoyl)-1H-pyrazol-4-yl]-aceticacid | —CH₂— | 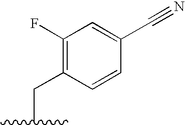 | H | Cl | Cl | H | 2.96 min.523 (M + H)⁺ |
| 4.10 | (1-(2-Chloro-phenyl)-5-(4-chloro-phenyl)-3-[(5-cyano-thiophen-2-ylmethyl)-carbamoyl]-1H-pyrazol-4-yl)-aceticacid | —CH₂— | 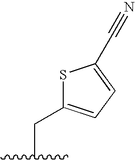 | H | Cl | Cl | H | 2.91 min.511 (M + H)⁺ |
| 4.11 | [1-(2-Chloro-phenyl)-3-(4-cyano-benzylcarbamoyl)-5-(4-fluoro-phenyl)-1H-pyrazol-4-yl]-aceticacid | —CH₂— | 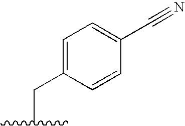 | H | F | Cl | H | 2.81 min.489 (M + H)⁺ |
| 4.12 | [5-(4-Chloro-phenyl)-3-(4-cyano-benzylcarbamoyl)-1-(2-fluoro-phenyl)-1H-pyrazol-4-yl]-aceticacid | —CH₂— | 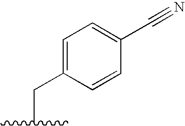 | H | Cl | F | H | 2.82 min.489 (M + H)⁺ |
| 4.13 | [5-(4-Chloro-phenyl)-1-(2-chloro-phenyl)-3-(3-cyano-benzylcarbamoyl)-1H-pyrazol-4-yl]-aceticacid | —CH₂— | 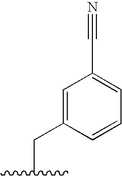 | H | Cl | Cl | H | 2.87 min.505 (M + H)⁺ |
| 4.14 | [5-(4-Chloro-phenyl)-1-(2-chloro-phenyl)-3-(4-cyano-phenylcarbamoyl)-1H-pyrazol-4-yl]-aceticacid | —CH₂— | 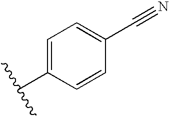 | H | Cl | Cl | H | 2.98 min.491 (M + H)⁺ |
| 4.15 | [5-(4-Chloro-phenyl)-1-(2-chloro-phenyl)-3-(3-cyano-phenylcarbamoyl)-1H-pyrazol-4-yl]-aceticacid | —CH₂— | 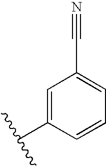 | H | Cl | Cl | H | 2.97 min.491 (M + H)⁺ |
| 4.16 | [5-(4-Chloro-phenyl)-1-(2-cyano-phenyl)-3-(4-fluoro-benzylcarbamoyl)-1H-pyrazol-4-yl]-aceticacid | —CH₂— | 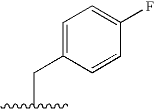 | H | Cl | CN | H | 2.78 min.489 (M + H)⁺ |
| 4.17 | [5-(4-Chloro-phenyl)-1-(2-chloro-phenyl)-3-(4-fluoro-phenylcarbamoyl)-1H-pyrazol-4-yl]-aceticacid | —CH₂— | 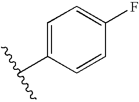 | H | Cl | Cl | H | 3.02 min.484 (M + H)⁺ |
| 4.18 | [1-(2,4-Dichloro-phenyl)-3-(4-fluoro-phenylcarbamoyl)-5-p-tolyl-1H-pyrazol-4-yl]-acetic acid | —CH₂— | 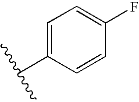 | H | —CH₃ | Cl | Cl | 3.19 min.499 (M + H)⁺ |
| 4.19 | [5-(4-Chloro-phenyl)-1-(2,4-dichloro-phenyl)-3-(4-sulfamoyl-benzylcarbamoyl)-1H-pyrazol-4-yl]-aceticacid | —CH₂— | 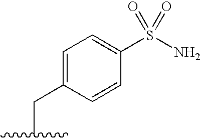 | H | Cl | Cl | Cl | 2.86 min.593 (M + H)⁺ |
| 4.20 | [5-(4-Chloro-phenyl)-1-(2,4-dichloro-phenyl)-3-(4-tetrazol-1-ylmethyl-phenylcarbamoyl)-1H-pyrazol-4-yl]-aceticacid | —CH₂— | 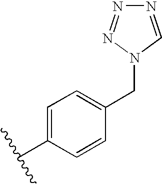 | H | Cl | Cl | Cl | 2.99 min.582 (M + H)⁺ |
| 4.21 | [3-(4-Cyano-benzylcarbamoyl)-1-(2,4-dichloro-phenyl)-5-p-tolyl-1H-pyrazol-4-yl]-acetic acid | —CH₂— | 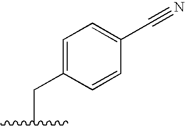 | H | —CH₃ | Cl | Cl | 3.05 min.519 (M + H)⁺ |
| 4.22 | [5-(4-Chloro-phenyl)-1-(2,4-dichloro-phenyl)-3-(4-fluoro-benzylcarbamoyl)-1H-pyrazol-4-yl]-aceticacid | —CH₂— | 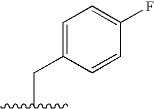 | H | Cl | Cl | Cl | 3.21 min.532 (M + H)⁺ |
| 4.23 | [5-(4-Chloro-phenyl)-3-(3-cyano-benzylcarbamoyl)-1-(2,4-dichloro-phenyl)-1H-pyrazol-4-yl]-aceticacid | —CH₂— | 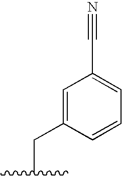 | H | Cl | Cl | Cl | 3.10 min.539 (M + H)⁺ |
| 4.24 | [5-(4-Chloro-phenyl)-3-(4-cyano-2-fluoro-benzylcarbamoyl)-1-(2,4-dichloro-phenyl)-1H-pyrazol-4-yl]-aceticacid | —CH₂— | 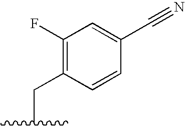 | H | Cl | Cl | Cl | 3.11 min.557 (M + H)⁺ |
| 4.25 | [5-(4-Chloro-phenyl)-1-(2,4-dichloro-phenyl)-3-(4-methoxy-benzylcarbamoyl)-1H-pyrazol-4-yl]-aceticacid | —CH₂— | 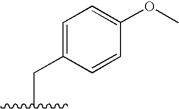 | H | Cl | Cl | Cl | 3.18 min.544 (M + H)⁺ |
| 4.26 | [5-(4-Chloro-phenyl)-1-(2,4-dichloro-phenyl)-3-(2-fluoro-benzylcarbamoyl)-1H-pyrazol-4-yl]-aceticacid | —CH₂— | 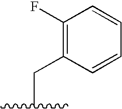 | H | Cl | Cl | Cl | 3.22 min.532 (M + H)⁺ |
| 4.27 | 3-Benzylcarbamoyl-5-(4-chloro-phenyl)-1-(2,4-dichloro-phenyl)-1H-pyrazol-4-yl]-aceticacid | —CH₂— | 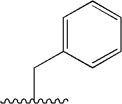 | H | Cl | Cl | Cl | 3.19 min.514 (M + H)⁺ |
| 4.28 | [5-(4-Chloro-phenyl)-1-(2,4-dichloro-phenyl)-3-(4-hydroxy-benzylcarbamoyl)-1H-pyrazol-4-yl]-aceticacid | —CH₂— | 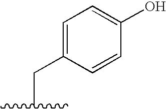 | H | Cl | Cl | Cl | 2.93 min.530 (M + H)⁺ |
| 4.29 | {5-(4-Chloro-phenyl)-1-(2,4-dichloro-phenyl)-3-[(thiophen-2-ylmethyl)-carbamoyl]-1H-pyrazol-4-yl}-acetic acid | —CH₂— | 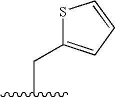 | H | Cl | Cl | Cl | 3.14 min.520 (M + H)⁺ |
| 4.30 | [5-(4-Chloro-phenyl)-1-(2,4-dichloro-phenyl)-3-(pyridin-3-ylcarbamoyl)-1H-pyrazol-4-yl]-aceticacid | —CH₂— | 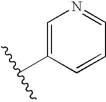 | H | Cl | Cl | Cl | 2.51 min.501 (M + H)⁺ |
| 4.31 | [5-(4-Chloro-phenyl)-3-(5-cyano-2-fluoro-benzylcarbamoyl)-1-(2,4-dichloro-phenyl)-1H-pyrazol-4-yl]-aceticacid | —CH₂— | 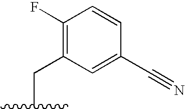 | H | Cl | Cl | Cl | 2.88 min.557 (M + H)⁺ |
| 4.32 | {5-(4-Chloro-phenyl)-1-(2,4-dichloro-phenyl)-3-[(furan-2-ylmethyl)-carbamoyl]-1H-pyrazol-4-yl}-acetic acid | —CH₂— | 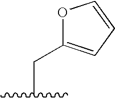 | H | Cl | Cl | Cl | 3.09 min.504 (M + H)⁺ |
| 4.33 | [1-(2-Chloro-phenyl)-5-(4-chloro-phenyl)-3-(3-cyano-4-fluoro-benzylcarbamoyl)-1H-pyrazol-4-yl]-aceticacid | —CH₂— | 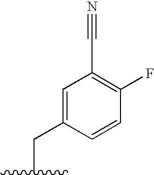 | H | Cl | Cl | H | 2.86 min.523 (M + H)⁺ |
| 4.34 | [5-(4-Chloro-phenyl)-1-(2,4-dichloro-phenyl)-3-(4-methoxy-benzylcarbamoyl)-1H-pyrazol-4-ylmethoxy]-acetic acid | —CH₂OCH₂— | 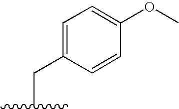 | H | Cl | Cl | Cl | 3.13 min.574 (M + H)⁺ |
| 4.35 | [5-(4-Chloro-phenyl)-1-(2,4-dichloro-phenyl)-3-(4-methoxy-benzylcarbamoyl)-1H-pyrazol-4-yloxy]-aceticacid | —OCH₂— | 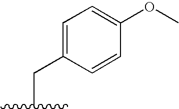 | H | Cl | Cl | Cl | 3.12 min.560 (M + H)⁺ |
| 4.36 | [5-(4-Chloro-phenyl)-1-(2,4-dichloro-phenyl)-3-(4-fluoro-benzylcarbamoyl)-1H-pyrazol-4-ylmethoxy]-acetic acid | —CH₂OCH₂— | 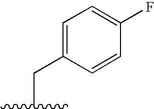 | H | Cl | Cl | Cl | 3.16 min.562 (M + H)⁺ |
| 4.37 | [5-(4-Chloro-phenyl)-3-(4-cyano-benzylcarbamoyl)-1-(2,4-dichloro-phenyl)-1H-pyrazol-4-ylmethoxy]-acetic acid | —CH₂OCH₂— | 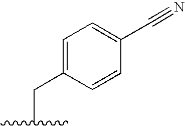 | H | Cl | Cl | Cl | 3.04 min.569 (M + H)⁺ |
| 4.38 | {5-(4-Chloro-phenyl)-1-(2-fluoro-phenyl)-3-[(4-fluoro-phenyl)-methyl-carbamoyl]-1H-pyrazol-4-yl}-acetic acid | —CH₂— | 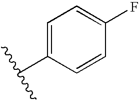 | —CH₃ | Cl | F | H | 2.74 min.482 (M + H)⁺Method (B) |
| 4.39 | [5-(4-Chloro-phenyl)-1-(2-chloro-phenyl)-3-(pyridin-3-ylcarbamoyl)-1H-pyrazol-4-yl]-aceticacid | —CH₂— | 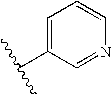 | H | Cl | Cl | H | 2.02 min.467 (M + H)⁺Method (B) |
| 4.40 | [5-(4-Chloro-phenyl)-1-(2-chloro-phenyl)-3-(2-piperidin-1-yl-ethyl-carbamoyl)-1H-pyrazol-4-yl]-acetic acid | —CH₂— | 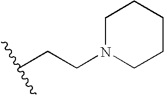 | H | Cl | Cl | H | 2.09 min.501 (M + H)⁺Method (B) |
| 4.41 | [5-(4-Chloro-phenyl)-3-cyclohexylcarbamoyl-1-(2-fluoro-phenyl)-1H-pyrazol-4-yl]-aceticacid | —CH₂— | 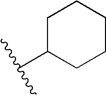 | H | Cl | Cl | H | 3.14 min.472 (M + H)⁺Method (B) |
| 4.42 | [5-(4-Chloro-phenyl)-1-(2-fluoro-phenyl)-3-(piperidin-1-ylcarbamoyl)-1H-pyrazol-4-yl]-aceticacid | —CH₂— | 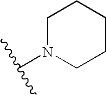 | H | Cl | Cl | H | 2.21 min.473 (M + H)⁺Method (B) |
| 4.43 | [3-(4-Carboxymethyl-phenylcarbamoyl)-5-(4-chloro-phenyl)-1-(2-fluorophenyl)-1H-pyrazol-4-yl]-aceticacid | —CH₂— | 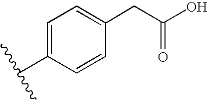 | H | Cl | Cl | H | 2.71 min.524 (M + H)⁺Method (B) |
| 4.44 | [3-(2-Benzylsulfanyl-ethylcarbamoyl)-5-(4-chloro-phenyl)-1-(2-fluorophenyl)-1H-pyrazol-4-yl]-aceticacid | —CH₂— | 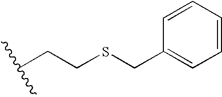 | H | Cl | Cl | H | 3.11 min.524 (M + H)⁺Method (B) |
| 4.45 | (5-(4-Chloro-phenyl)-1-(2-fluoro-phenyl)-3-(2-methylamino-ethylcarbamoyl)-1H-pyrazol-4-yl]-aceticacid | —CH₂— | Methyl-amino-ethyl | H | Cl | Cl | H | 1.85 min.447 (M + H)⁺Method (B) |

Synthesis:

### Compound 4.01

Prepared according to the procedure outlined in scheme 9:

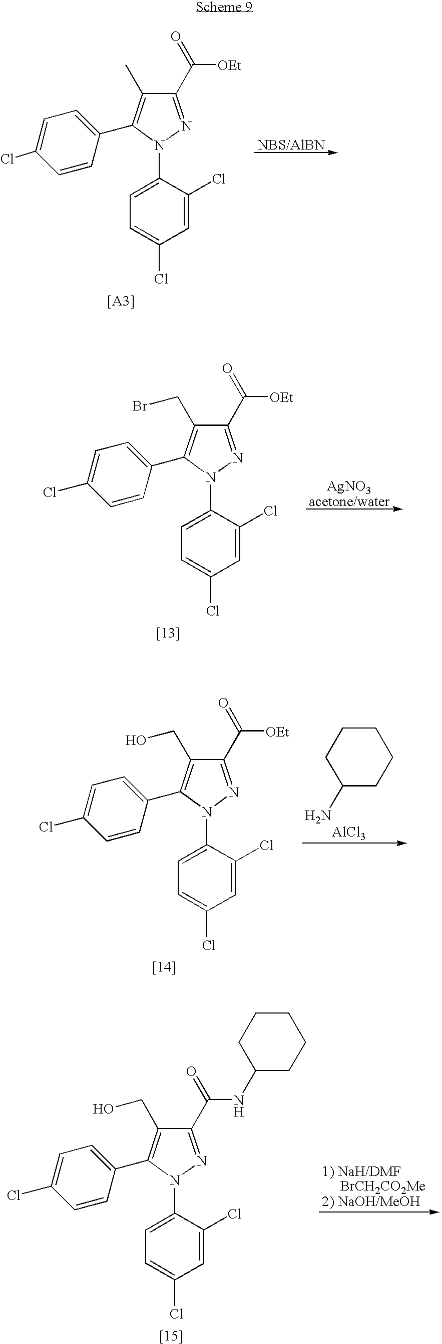

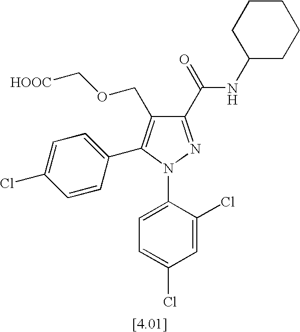

5-(4-Chloro-phenyl)-1-(2,4-dichloro-phenyl)-4-methyl-1H-pyrazole-3-carboxylic acid ethyl ester [A3] was prepared according to the procedure outlined in scheme 1.

A solution of [A3] (2.08 g, 5.08 mmol), N-bromosuccinimide (0.994 g, 5.60 mmol) and 1,1′-azobis(isobutyronitrile) (0.009 g, 0.05 mmol) in carbon tetrachloride (30 ml) was heated at reflux for 12 hours. The mixture was evaporated and the residue purified by chromatography over silica, eluting with ethyl acetate/cyclohexane (1/4) to give 4-bromomethyl-5-(4-chloro-phenyl)-1-(2,4-dichloro-phenyl)-1H-pyrazole-3-carboxylic acid ethyl ester [13] (1.5 g, 3.07 mmol) as an oil.

Compound [13] (1.5 g, 3.07 mmol) was dissolved in acetone/water (2:1, 10 ml) and added to a solution of silver nitrate (1.8 g, 10.7 mmol) in acetone/water (1:1, 70 ml) and the resulting solution was heated at 60° C. for 16 hours (see US patent application N° 2004/192667). The mixture was filtered, acetone removed by evaporation and the solution extracted with dichloromethane. Organic extracts were dried over sodium sulphate, filtered and evaporated to give 5-(4-chloro-phenyl)-1-(2,4-dichloro-phenyl)-4-hydroxymethyl-1H-pyrazole-3-carboxylic acid ethyl ester [14] (1.01 g, 2.37 mmol) as a solid.

A solution of cyclohexylamine (1.1 ml, 9.6 mmol) in dichloromethane (2 ml) was added to a stirred solution of aluminium trichloride (0.633 g, 4.7 mmol) in dichloroethane (10 ml) at 0° C. The mixture was warmed to 20° C. and a solution of compound [14] (1.01 g, 2.4 mmol) in dichloromethane (3 ml) was added. After 4 hours, water (5 ml) was added and the mixture stirred for 30 min and then filtered through celite. Further water was added and the mixture extracted with dichloromethane. Organic extracts were dried over sodium sulphate, filtered and evaporated to give 5-(4-chloro-phenyl)-1-(2,4-dichloro-phenyl)-4-hydroxymethyl-1H-pyrazole-3-carboxylic acid cyclohexylamide [15](1.07 g, 2.2 mmol).

Sodium hydride (60%, 72 mg, 1.8 mmol) was added to a stirred solution of compound [15] (0.434 g, 0.91 mmol) in N,N-dimethylformamide (8 ml) at 20° C. After 30 minutes, bromo-acetic acid methyl ester (0.17 ml, 1.8 mmol) was added and the solution was stirred at 20° C. for 4 hours then at 60° C. for 2 hours. The mixture was cooled to 20° C., diluted with water and extracted with ethyl acetate. Organic extracts were dried over sodium sulphate, filtered and evaporated. The residue was purified by chromatography over silica, eluting with ethyl acetate/cyclohexane (1/4). The product was dissolved in methanol (6 ml) and potassium hydroxide (0.11 g, 1.94 mmol) was added. After stirring for 2 hours, the mixture was acidified with 6M hydrochloric acid and extracted with dichloromethane. Organic extracts were dried over sodium sulphate, filtered and evaporated. The residue was purified by chromatography over silica, eluting with ethanol/dichloromethane (19/1) followed by trituration with ether to give the title compound [4.01] (0.022 g, 0.041 mmol).

¹H NMR (DMSO-D₆): δ 1.20-1.45 (6H, m), 1.70-1.85 (4H, m), 3.72-3.82 (1H, m), 4.07 (2H, s), 4.68 (1H, s), 7.37 !H, d), 7.48 (1H, d), 7.63 (1H, dd), 7.82 (1H, d), 7.83 (1H, d), 8.09 (1H, d).

Compounds 4.34, 4.36 and 4.37 were obtained in an analogous manner to compound 4.01

### Compound 4.02

Prepared according to the procedure outlined in scheme 10:

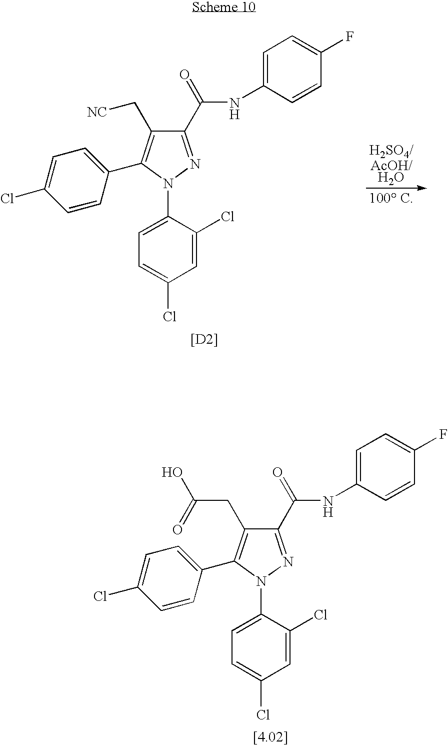

5-(4-Chloro-phenyl)-4-cyanomethyl-1-(2,4-dichloro-phenyl)-1H-pyrazole-3-carboxylic acid (4-fluoro-phenyl)-amide [D2] was obtained according to the procedure outlined in scheme 6.

Compound [D2] (0.12 g, 0.24 mmol) was dissolved in a mixture of concentrated sulphuric acid (0.2 ml), water (0.6 ml) and acetic acid (2 ml). The resulting solution was heated at 100° C. for 16 hours then poured into ice/water and the mixture extracted with ethyl acetate. Organic extracts were dried over sodium sulphate and evaporated. The residue was purified by chromatography over silica, eluting with ethyl acetate to give the title compound [4.02] (0.006 g, 0.012 mmol, 50%) as a white solid.

¹H NMR (CDCl₃): δ3.74 (2H, s), 7.12 (2H, t), 7.24-7.31 (4H, m), 7.34-7.41 (3H, m), 7.52 (1H, d), 7.67 (1H, dd), 8.93 (1H, s, br).

### Compound 4.30

Prepared according to the procedure outlined in scheme 11:

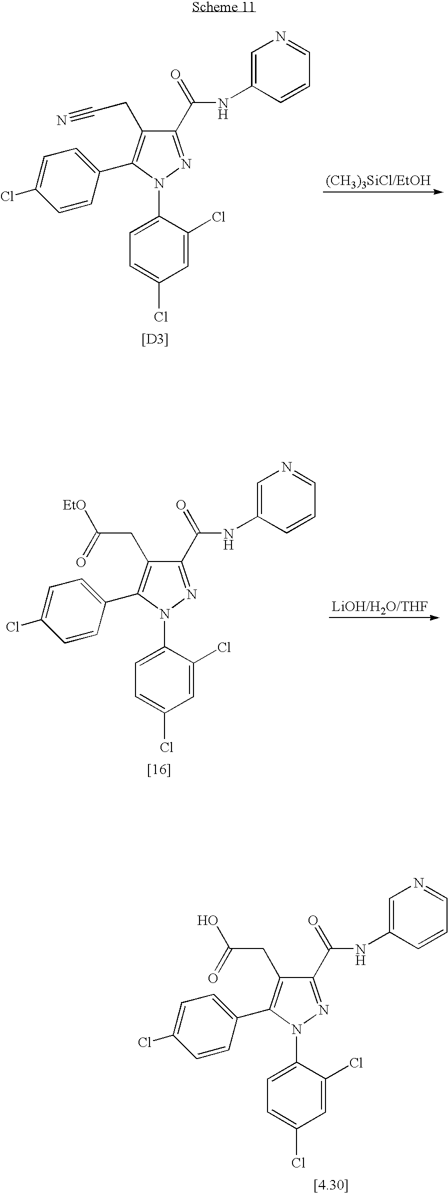

5-(4-Chloro-phenyl)-4-cyanomethyl-1-(2,4-dichloro-phenyl)-1H-pyrazole-3-carboxylic acid pyridin-3-ylamide [D3] was obtained according to the procedure outlined in scheme 6.

Chlorotrimethylsilane (0.30 ml, 2.6 mmol) was added to a solution of [D3] (0.157 g, 0.33 mmol) in ethanol (3 ml). The resulting solution was heated to 50° C. for 24 hours with the addition of further chlorotrimethylsilane (1.12 ml, 9.9 mmol) during this time. Water was added, the mixture made basic by addition of potassium carbonate and extracted with dichloromethane. The organic extracts were dried over sodium sulphate, filtered and evaporated to give crude compound [16] (0.215 g).

A solution of lithium hydroxide (0.051 g, 1.2 mmol) in water was added to a stirred solution of compound [16] (0.215 g) in tetrahydrofuran/water (1/1) at 20° C. After 16 hours, water was added and the mixture acidified then extracted with ethyl acetate. Organic extracts were dried over sodium sulphate and evaporated. The residue was purified by chromatography over silica, eluting with a cyclohexane/ethyl acetate/acetic acid gradient to give the title compound [4.30] (0.006 g, 0.012 mmol, 10%) as a white solid.

¹H NMR (DMSO-D₆): δ 3.56 (2H, s), 7.33 (2H, d), 7.38 (1H, dd), 7.49 (2H, d), 7.62 (1H, dd), 7.81-7.84 (2H, m), 8.24 (1H, dt), 8.30 (1H, dd), 8.99 (1H, d).

Compounds 4.03, 4.17-4.20, 4.22, 4.25-4.30, 4.32, 4.34-4.36 and 4.38-4.44 were obtained in an analogous manner to either [4.2] or [4.30]. Compound 4.45 was obtained in an analogous manner to [4.30] but with (2-amino-ethyl)-methyl-carbamic acid tert-butyl ester as amine A₂R₂NH₂ whereby the tert.-butyloxycarbonyl group was removed during the nitrile hydrolysis step.

### Compound 4.04

Prepared according to the procedure outlined in scheme 12:

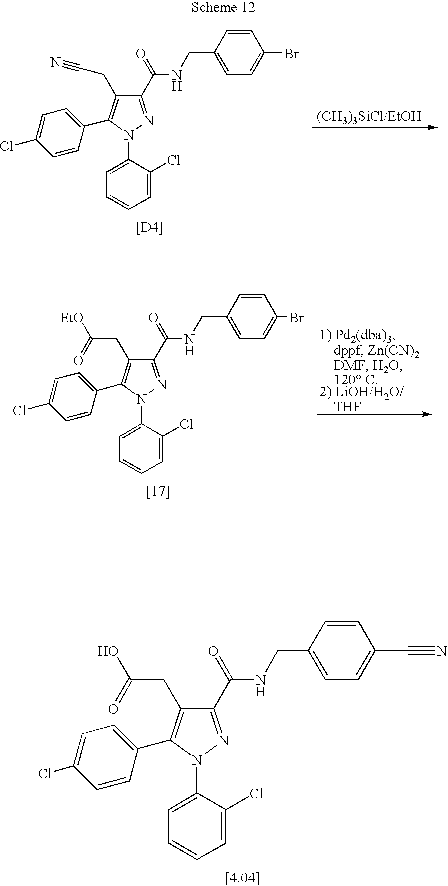

1-(2-Chloro-phenyl)-5-(4-chloro-phenyl)-4-cyanomethyl-1H-pyrazole-3-carboxylic acid 4-bromo-benzylamide [D4] was obtained according to the procedure outlined in scheme 6.

To a solution of [D4] (0.67 g, 1.24 mmol) in ethanol (8 ml) was added chlorotrimethylsilane (2.0 ml, 15.7 mmol). The reaction mixture was stirred for 15 hours at 60° C. and then evaporated under reduced pressure. The residue was then diluted with a saturated aqueous sodium bicarbonate solution and extracted with ethyl acetate. The combined organic layers were dried over sodium sulphate, filtered and evaporated to give [3-(4-bromo-benzylcarbamoyl)-1-(2-chloro-phenyl)-5-(4-chloro-phenyl)-1H-pyrazol-4-yl]-acetic acid ethyl ester [17] (0.605, 1.03 mmol).

A solution of compound [17] (0.605 g, 1.03 mmol), tris(dibenzylidineacetone)dipalladium (0) [Pd₂(dba)₃](0.11 g, 0.12 mmol), 1,1′-bis(diphenylphosphino)ferrocene (dppf)(0.167 g, 0.3 mmol) and zinc cyanide (0.199 g, 1.7 mmol) in a mixture of N,N-dimethylformamide (8 ml) and water (0.8 ml) was degassed with argon under sonication for 5 min. Then the mixture was heated to 120° C. for 3 hours then cooled to 20° C. and stirred for a further 15 hours. The crude mixture was filtered through a pad of silica gel and evaporated. The residue was purified by chromatography over silica, eluting with an ethyl acetate/cyclohexane mixture. The resulting product was dissolved in tetrahydrofuran (5 ml) and a solution of lithium hydroxide hydrate (0.028 g, 0.68 mmol) in water (5 ml) was added. After 2 hours at 20° C., the mixture was acidified with 2M hydrochloric acid and extracted with ethyl acetate. Organic extracts were dried over sodium sulphate, filtered and evaporated. The residue was purified by chromatography over silica, eluting with ethyl acetate/cyclohexane (1/1) containing acetic acid (1%) to give the title compound [4.04] (0.087 g, 0.16 mmol, 16%).

¹H NMR (CDCl₃): δ3.72 (2H, s), 4.73 (1H, d), 7.23 (2H, d), 7.28-7.36 (4H, m), 7.41 (1H, dt), 7.46-7.50 (3H, m), 7.65 (2H, d), 7.79 (1H, t).

Compounds 4.05-4.09, 4.11-4.15, 4.21, 4.23, 4.24, 4.31, 4.33 and 4.37 were obtained in an analogous manner to compound 4.04.

### Compound 4.10

(5-Bromo-thiophen-2-yl)-methylamine trifluoroacetate salt [17] was obtained according to the procedure outlined in scheme 13:

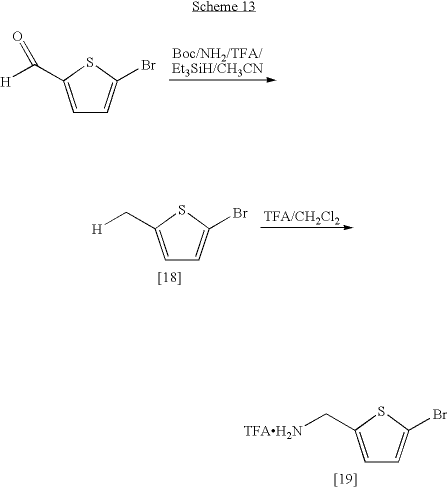

To a solution of 5-bromothiophene-2-carboxaldehyde (4.7 g, 25 mmol) in acetonitrile (80 ml) at 20° C. was added tert-butylcarbamate (3.5 g, 30.4 mmol), triethylsilane (6.0 ml, 37.5 ml) and trifluoroacetic acid (2.8 ml, 37.5 mmol). The resulting mixture was stirred for 16 hours at 20° C., then hydrolysed with a saturated aqueous sodium bicarbonate solution and extracted with ethyl acetate. The combined organic layers were dried over sodium sulphate, filtered and evaporated. The residue was purified by chromatography over silica, eluting with cyclohexane/ethyl acetate (9/1) to give 2-bromo-5-methyl-thiophene [18] (3.4 g, 11.6 mmol) as an oil.

Compound [19] was obtained by treatment of [18] with trifluoroacetic acid in dichloromethane followed by evaporation of the mixture to dryness.

Compound [4.10] was obtained following the procedure outlined in scheme 14:

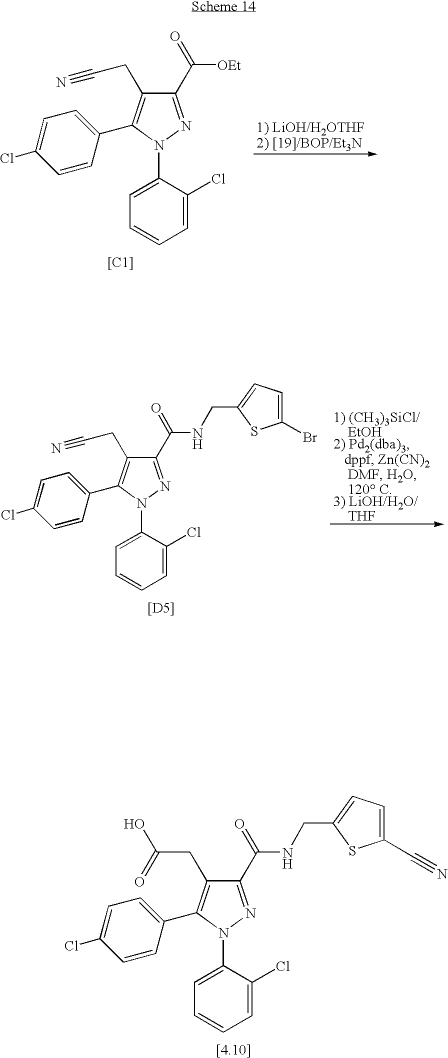

1-(2-Chloro-phenyl)-5-(4-chloro-phenyl)-4-cyanomethyl-1H-pyrazole-3-carboxylic acid ethyl ester [C1] was obtained following the procedure outlined in scheme 6. Hydrolysis of [C1] followed by coupling with amine [17] according to procedures previously described in scheme 6, gave 1-(2-chloro-phenyl)-5-(4-chloro-phenyl)-4-cyanomethyl-1H-pyrazole-3-carboxylic acid (5-bromo-thiophen-2-ylmethyl)-amide [D5].

Intermediate [D5] (1.23 g, 2.25 mmol) was subjected to an analogous reaction sequence as previously outlined in scheme 12 for the synthesis of [4.4] to give the title compound [4.10] (0.45 g, 0.88 mmol, 39%) as a foam.

¹H NMR (CDCl₃): δ3.71 (2H, s), 4.88 (2H, d), 7.10 (1H, d), 7.11-7.29 (4H, m), 7.34 (2H, d), 7.43 (1H, t), 7.48 (1H, d), 7.53 (1H, d), 7.75 (1H, t).

Compound 4.16 was obtained from 1-(2-bromo-phenyl)-5-(4-chloro-phenyl)-4-cyanomethyl-1H-pyrazole-3-carboxylic acid 4-fluorobenzylamide [D6] using an analogous procedure to the above and as outlined in scheme 15:

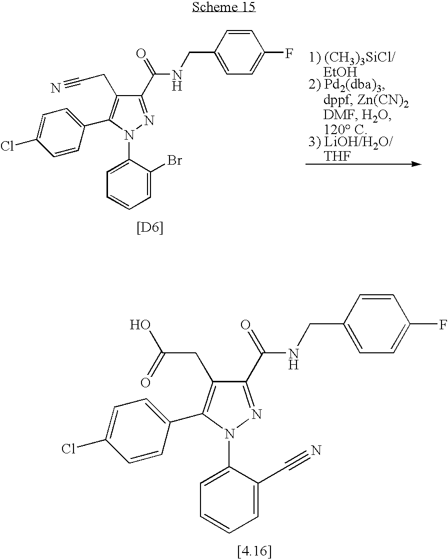

### Compound 4.35

Prepared according to the procedure outlined in scheme 16:

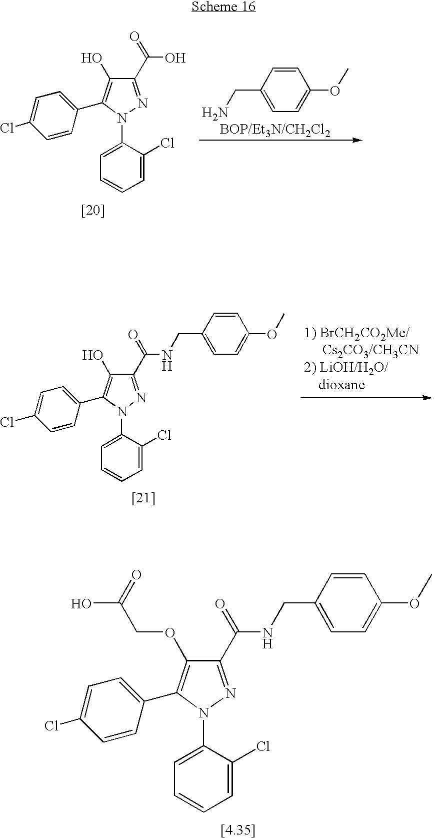

5-(4-Chloro-phenyl)-1-(2-chloro-phenyl)-4-hydroxy-1H-pyrazole-3-carboxylic acid [20] was obtained according to the procedure published for related compounds in WO/2006035310. This compound was coupled with 4-methoxybenzylamine according to the procedure previously outlined in scheme 1, to give 5-(4-chloro-phenyl)-1-(2-chloro-phenyl)-4-hydroxy-1H-pyrazole-3-carboxylic acid 4-methoxy-benzylamide [21].

Cesium carbonate (0.11 g, 0.34 mmol) followed by bromoacetic acid methyl ester (0.04 ml, 0.34 mmol) were added to a stirred solution of compound [21] 0.14 g, 0.28 mmol) in acetonitrile (5 ml) at 20° C. After 15 hours, the mixture was filtered and evaporated. The residue was dissolved in 1,4-dioxane (5 ml) and a solution of lithium hydroxide hydrate (0.036 g, 0.84 mmol) in water (5 ml) was added. After 2 hours at 20° C., the mixture was acidified with 1M hydrochloric acid and extracted with ethyl acetate. Silica was added to the organic extracts, solvent evaporated and the residual powder added to a silica column. The column was eluted with ethyl acetate/acetic acid (99/1) and product-containing fractions evaporated to give the title compound [4.35] (0.086 g, 0.15 mmol, 53%).

¹H NMR (CDCl₃): δ3.82 (3H, s), 4.40 (2H, s), 4.60 (2H, d), 6.90 (2H, d), 7.17 (2H, d), 7.26 (1H, t, br), 7.30 (2H, d), 7.34 (1H, d), 7.36 (2H, d), 7.38 (1H, d), 7.47 (1H, d).

### Biological Activities

### A. Receptor In Vitro Assay

CP55940 disclosed by Felder et al., 1995, Molecular Pharmacology, 48, 443 is a known CB1 receptor agonist. The receptor functional activities of the above Examples of compounds of the invention were evaluated in vitro by measuring their ability to inhibit CP55940-induced GTPγS-Eu binding to membranes prepared from Sf9 cells expressing the human CB1 receptor and the G protein alphai3, beta1 and gamma2 subunits (CB1αi3β1γ2).

### GTPγS-Eu Binding Assay

The GTPγS-Eu binding assay was conducted in a 96-well Acrowell plate, using the Delfia GTP-binding kit (PerkinElmer Life Sciences), and according to the manufacturer's instructions. Briefly, cell membranes (5-10 μg), obtained from PerkinElmer Life Sciences, were incubated in triplicate with 3 μM GDP and test compounds in 100 μl buffer (50 mM HEPES, 10 mM MgCl₂, 100 mM NaCl, supplemented with 300 μg/ml saponin) for 40 min at room temperature on an orbital shaker. 10 μl of GTPγS-Eu was then added to a final concentration of 9 nM and the incubation continued for a further 30 min. Samples were then harvested under vacuum and the plate was washed twice with 300 μl of wash buffer. The fluorescence was read immediately using the Envision plate reader (PerkinElmer Life Sciences) on a Delfia module. Basal levels of GTPγS-Eu binding were defined as that in the absence of compound.

Data were analysed using the computer program GraphPad Prism (GraphPad Software Inc.). First, a concentration-response curve for the reference agonist CP55940 was generated and analysed by non linear least squares regression analysis to define its EC₅₀ value. CP55940 induced a concentration-dependant stimulation of GTPγS-Eu binding to CB1αi3β1γ2 membranes with an average EC₅₀ of 2 nM. A single concentration of CP55940 (10 nM) was then used to stimulate the binding of GTPγS-Eu to CB1αi3β1γ2 membranes and various concentrations of the compounds of the invention were used to inhibit that stimulation. IC₅₀ values for compounds of the invention were determined and their inhibition constants (Ki) were calculated using the equation of Cheng and Prusoff (Biochem. Pharmacol., 22, 3099-3108, 1973). This equation is well known in the art. The dissociation constant of the reference agonist CP55940 used in the equation is that given by PerkinElmer Life Science for each batch of CB1/2αi3β1γ2 membranes.

Most of the Example compounds of the invention had Ki values of less than 1 μM. in the above assay.

### B. In Vivo Model Measuring Effects on Body Weight

The effects of chronic administration of test compounds on body weight and white adipose tissue mass (epididymal) were studied in male Swiss CD-1 mice (7-8 weeks old at entrance).

A habituation of high fat diet regimen (42% of kcal derived from fat) is performed 3 days before the initiation of the treatment. On test day 1 (before treatment initiation) animals are weighed and allocated into weight-matched treatment groups. 10 mice (5 mice/cage) are used for each group. All along the treatment period, high fat food and drinking water are provided ad libitum. Animals are maintained on a normal phase 12 hour light-dark cycle.

Test compounds are dissolved in vehicle: 1% HPMC (hydroxyl propyl methyl cellulose), 0.1% Tween 80, 1% DMSO and dosed orally once daily by gavage with an administration volume of 5 ml/kg. Body weight of each mouse is recorded daily. After 21 consecutive days of treatment, mice are sacrificed by cervical dislocation and white adipose tissue (epididymal) of each mouse is removed and weighed.

Effect of chronic administration of test compounds is expressed as absolute body weight gain at day 21 and as proportion of white adipose tissue mass normalised to body weight at day 21.

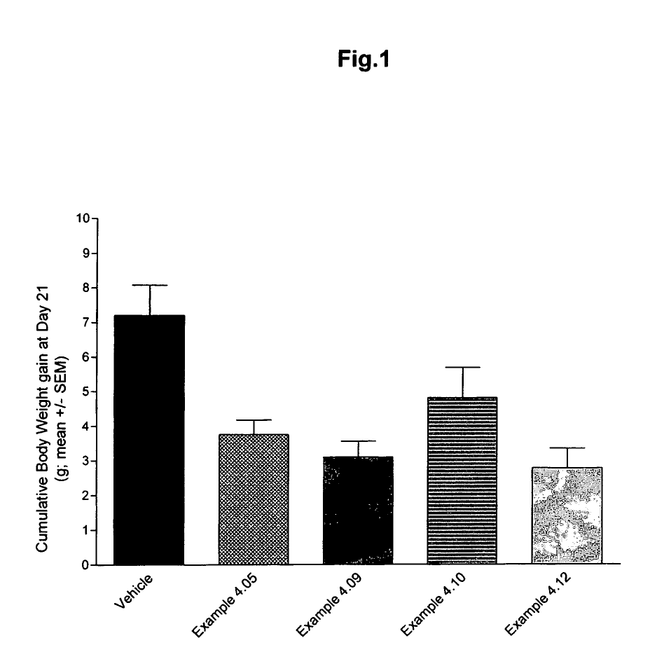

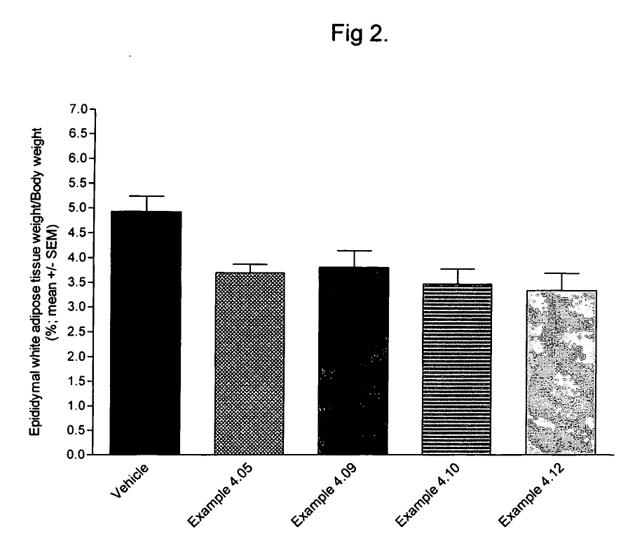

FIGS. 1 and 2 show the effect of chronic treatment of test compounds on absolute body weight gain and on white adipose tissue mass normalised to body weight, respectively. The test compounds, Example 4.05, Example 4.09, Example 4.10 and Example 4.12, are administrated orally once daily (10 mg/kg) for 21 consecutive days.

### C. In Vivo Model Measuring Effect on Gastrointestinal Transit

The effects of test compounds on the gastrointestinal transit were studies in male Swiss CD-1 mice (6-10 weeks old at entrance). The cannabinoid CB1 receptor has been implicated in the control of the gastrointestinal transit in rodents. Stimulation with a CB1 agonist decreases the transit time of the gastrointestinal tract whereas a CB1 antagonist has the opposite effect. Reports in literature suggests that this action is primarily mediated via peripheral CB1 receptors in the gut (eg Casu et al., European Journal of Pharmacology, 459 (2003):97-105).

6 mice (2-6 mice/cage) are used for each treatment group. Animals are maintained on a normal phase 12 hour light-dark cycle. Food (standard chow) and water are provided ad libitum, unless otherwise stated. Test compounds are dissolved in vehicle: 1% HPMC (hydroxylpropyl methyl cellulose), 0.1% Tween 80, 1% DMSO and dosed orally by gavage with an administration volume of 5 ml/kg.

Mice are fasted 18 hours before conducting the gastrointestinal transit test.

Test compounds are administered per os 45 min before the administration of 10% charcoal, a none absorbed black marker. The charcoal is dosed with an administration volume of 10 ml/kg orally by gavage. 5% Gum arabic is used as solvent for charcoal
Twenty min after the charcoal meal, mice are killed by cervical dislocation and intestines are removed from the pylorus to the cecum. The distance covered by the head of the marker is measured and expressed as percent of the total length of the small intestine.

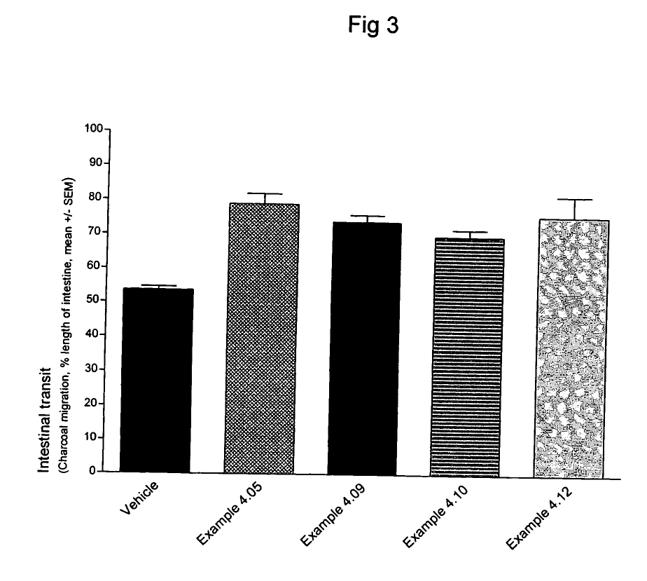

FIG. 3 shows the effect on gastrointestinal transit after oral administration (20 mg/kg) of test compounds: Example 4.05, Example 4.09, Example 4.10 and Example 4.12.

1. A compound of formula (I), or a salt, hydrate, solvate or N-oxide thereof: 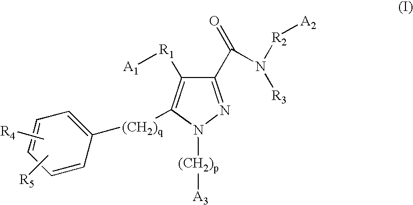
wherein
A₁ is hydrogen, —COOH, or tetrazolyl, and A₂ is hydrogen, —COOH, tetrazolyl, —CN, —CF₃, —COR₆, —SO₂R₆, —OR₇, —NR₇R₈, —NHCOR₆, or —NR₇SO₂R₈ provided that one of A₁ and A₂ is either
—COOH or tetrazolyl;
p is 0 or 1 and A₃ is phenyl or cycloalkyl, either of which is optionally substituted with R₄ and/or R₅;
q is 0 or 1;
R₁ is a bond, or —(CH₂)ₐB₁(CH₂)_{b}— wherein a and b are independently 0, 1, 2 or 3 provided that a+b is not greater than 4, and B₁ is —CO—, —O—, —S—, —SO—, —SO₂—, —CH₂—, —CHOH— or —NR₇—.
R₂ is a bond, —(CH₂)ₐB₁(CH₂)_{b}— or —[(CH₂)ₐB₁(CH₂)_{b}]ₙ-A₄-[(CH₂)_{c}B₂(CH₂)d]ₘ— wherein a, b, and B₁ are as defined for R₁; B₂ is as defined for B₁, c and d are independently 0, 1, 2 or 3; with the proviso that a+b+c+d is not greater than 6, n and m are independently 0 or 1 and A₄ is a monocarbocyclic or monoheterocyclic ring, having 3 to 8 ring atoms, optionally substituted with one or more of —F, —Cl, —Br, —CN, —CF₃, C₁-C₄ alkyl, cycloalkyl, —OR₉, oxo or —NR₇R₈;
R₃ is hydrogen, C₁-C₄ alkyl, cycloalkyl, —CF₃, —OR₉, —NR₇R₈, —(CH₂)ₛCOR₆, —(CH₂)ₛSO₂R₆, —(CH₂)ₛNR₇COR₆, —(CH₂)ₛNR₇COOR₈, —(CH₂)ₛNR₇SO₂R₆, wherein s is 1, 2, 3 or 4;
R₄ and R₅ independently —R₉, —CN, —F, —Cl, —Br, —OR₉, —NR₇R₈, —NR₇COR₆, —NR₇SO₂R₆, —COR₆, —SR₉, —SOR₉, —SO₂R₆, (C₁-C₄ alkyl)OR₉, —(C₁-C₄ alkyl)NR₇R₈, —(C₁-C₄ alkyl)NR₇COR₆, C₁-C₄ alkyl)NR₇COOR₈, —(C₁-C₄ alkyl)NR₇SO₂R₆, —(C₁-C₄ alkyl)COR₆, —(C₁-C₄ alkyl)SO₂R₆, —NR₇COOR₈, or [N—(C₁-C₄ alkyl)]-tetrazolyl;
R₆ is C₁-C₄ alkyl, cycloalkyl, —CF₃ or —NR₇R₈;
R₇ and R₈ are independently hydrogen, C₁-C₄ alkyl or cycloalkyl; and
R₉ is hydrogen, C₁-C₄ alkyl, cycloalkyl, fully or partially fluorinated C₁-C₄ alkyl.

2. A compound as claimed in claim 1 wherein p is 0.

3. A compound as claimed in claim 1 wherein any optional R₄ and/or R₅ substituents in A₃ are selected from —Cl, —F, —Br, —CN, —CF₃, C₁-C₄ alkyl, cycloalkyl, —O(C₁-C₄ alkyl), —O(cycloalkyl), —SO₂(C₁-C₄ alkyl), —SO₂NH(cycloalkyl), —SO₂NH(C₁-C₄ alkyl), —SCF₃ and —OCF₃.

4. A compound as claimed in claim 1 wherein A₃ is a phenyl ring.

5. A compound as claimed in claim 4 wherein the phenyl ring A₃ is substituted in the ortho position and/or the para position by —Cl, —CF₃, —OCF₃, —SCF₃, —F, —Br, or —CN.

6. A compound as claimed in claim 1 wherein q is 0.

7. A compound as claimed in claim 1 wherein, in the phenyl ring shown in formula (I), any optional R₄ and/or R₅ substituents are selected from —Cl, —F, —Br, —CN, —CF₃, C₁-C₄ alkyl, cycloalkyl, —SO₂R₆, —OR₉ and —SR₉.

8. A compound as claimed in claim 1 wherein, in the phenyl ring shown in formula (I), R₄ is hydrogen and R₅ is a substituent in the para position selected from —Cl, —F, —Br, —CN, —CF₃, C₁-C₄ alkyl, cycloalkyl, —O(C₁-C₄ alkyl), —O(cycloalkyl), —OCF₃, —SO₂(C₁-C₄ alkyl), —SO₂NH₂, —SO₂NHCH₃, and —SO₂N(CH₃)₂.

9. A compound as claimed in claim 1 wherein R₃ is hydrogen, methyl or methoxy.

10. A compound as claimed in claim 1 wherein A₂ is tetrazolyl or —COOH, and A₁ is hydrogen.

11. A compound as claimed in claim 10 wherein R₂ is a bond, or —(CH₂)₁₋₅—.

12. A compound as claimed in claim 10 wherein —R₂A₂ is

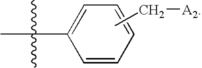

13. A compound as claimed in claim 10 wherein R₁ is —CH₂—.

14. A compound as claimed in claim 1 wherein A₁ is tetrazolyl or —COOH, and A₂ is hydrogen, —COR₆, —OR₇, —NR₇R₈, —NHCOR₆, —CN, —CF₃, —SO₂N(R₇)(R₈), —NR₇SO₂CF₃, or —NR₇SO₂R₇.

15. A compound as claimed in claim 1 wherein A₁ is tetrazolyl or —COOH, and —R₂A₂ is —OCH₂COOH.

16. A compound as claimed in claim 14 wherein R₁ is —(CH₂)ₐB₁(CH₂)_{b}— wherein a and b are each 1.

17. A compound as claimed in claim 14 wherein R₁ is —CH₂— or —OCH₂—.

18. A compound as claimed in claim 1 which has formula (IA): 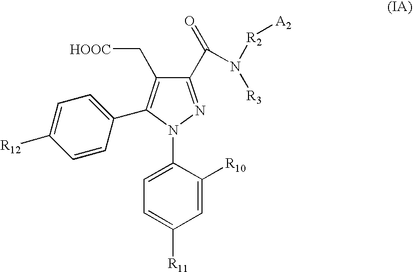
wherein R₁₀ is —Cl, —CF₃, —OCF₃, —F, —Br, or —CN; R₁₁ is hydrogen, —Cl, —CF₃, —OCF₃, —F, —Br, or —CN; R₁₂ is —Cl, —CF₃, —OCF₃, —F, —Br, —OCH₃, —O(cyclopropyl), —SO₂NH₂, or —CN.

19. A compound as claimed in claim 14 wherein, when A₂ is not hydrogen, any R₆, R₇, and R₈ present in A₂ are independently selected from hydrogen, methyl, —SO₂NH₂, —SO₂NHCH₃, —CONHCH₃, 2-oxo-pyrrolidin-1-yl, or 2-oxo-piperidin-1-yl.

20. A compound as claimed in claim 14 wherein A₂ is —CONH₂, —OCH₃ or —NHSO₂CH₃.

21. A compound as claimed in claim 14 wherein R₂ is a bond, or —(CH₂)₁₋₅— or the radical —R₂A₂ is

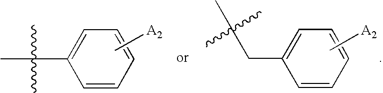

22. A compound as claimed in claim 21 wherein A₂ is in the 4-position of the phenyl ring.

23. A compound as claimed in claim 14 wherein R₂ is —[(CH₂)ₐB₁(CH₂)_{b}]ₙ-A₄-[(CH₂)_{c}B₂(CH₂)d]ₘ— wherein A₄ is a divalent piperidine, piperazine, piperazine optionally substituted by methyl on one of the ring nitrogens, morpholine, cyclopropane, cyclobutane, cyclopentane, cyclohexane, cycloheptane, or 2-oxo-pyrrolidine radical.

24. A compound as claimed in claim 14 wherein R₂ is —[(CH₂)ₐB₁(CH₂)_{b}]ₙ-A₄-[(CH₂)_{c}B₂(CH₂)d]ₘ— wherein A₄ is a divalent phenylene or monocyclic heteroarylene radical having from 5 or 6 ring atoms, optionally substituted with R₄ and/or R₅.

25. A compound as claimed in claim 14 wherein R₂ is —(CH₂)ₐB₁(CH₂)_{b}.

26. A compound as claimed in any of claims claim 23 wherein A₂ is hydrogen.

27. A compound as claimed in claim 23 wherein, in the radical R₂, m is 0 and n is 0.

28. A compound as claimed in claim 23 wherein, in the radical R₂, m is 1 and n is 0.

29. A compound as claimed in claim 23 wherein, in the radical R₂, n is 1 and m is 0.

30. A compound as claimed in claim 23 wherein, in the radical R₂, n is 1 and m is 1.

31. A compound as claimed in claim 24 wherein, in the radical R₂, B₁ and/or B₂ are each —CH₂—, and a and b, or c and d, or a, b, c and d are each 0.

32. A compound as claimed in claim 29 wherein, in the radical R₂, a and b are each independently 1 or 2; or c and d are each independently 1 or 2; or a, b, c and d are each independently 1 or 2.

33. A pharmaceutical composition comprising a compound as claimed in claim 1.

34-35. (canceled)

36. A method for the treatment of treatment of diseases or pathologic conditions associated with metabolic disorders mediated by or related to CB1 mediated mechanisms, which method comprises administering to a subject suffering such disease or condition an effective amount of a compound as claimed in claim 1.

37. A method as claimed in claim 36 wherein the disease or condition treated is obesity or overweight, or a disease or condition related to obesity or overweight.
